(19) United States
(12) Patent Application Publication
(10) Pub. No.: US 2020/0141925 A1
(43) Pub. Date: May 7, 2020

(54) Risk Assessment Tool for Patients with Sepsis

(71) Applicant: McMaster University, Hamilton (CA)

(72) Inventors: Patricia Liaw, Dundas (CA); Alison Fox- Robichaud, Hamilton (CA); Ponnambalam Ravi Selvaganapathy, Dundas (CA); Dhruva Dwivedi, Milton (CA); Ellen McDonald, Caledonia (CA); Kao-lee Liaw, Dundas (CA)

(21) Appl. No.: 16/629,204

(22) PCT Filed: Jul. 9, 2018
(86) PCT No.: PCT/CA2018/050833

§ 371 Date: Jan. 7, 2020

Related U.S. Application Data
(60) Provisional application No. 62/529,767, filed on Jul. 7, 2017.

(51) Int. Cl. G01N 33/50; G01N 33/68; G01N 33/70; C12Q 1/6876; G16H 50/30

(52) U.S. Cl. CPC G01N 33/5091; G01N 33/6893; G01N 33/70; C12Q 1/6876; G01N 2800/52; G01N 2800/26; G01N 2800/56; C12Q 2600/118; C12Q 2600/158; G16H 50/30

(57) ABSTRACT

The present invention provides a prognostic and mortality risk assessment method for patients with sepsis. The method involves measuring a combination of cell-free DNA (cfDNA), protein C, lactate, platelet count, creatinine level, and Glasgow Coma Score (GCS) and analyzing the measured values using a complementary log-log model to determine the daily and 28-day (or other fixed term) probabilities of dying for septic patients and a binomial logit model to distinguish septic patients from non-septic patients.

### FIELD OF THE INVENTION

The present application relates to a prognostic method in the field of infectious disease and critical care, and in particular, to a method of assessing the risk of death in patients with sepsis.

### BACKGROUND OF THE INVENTION

Sepsis (or “blood poisoning”) is a life-threatening condition characterized by systemic inflammation and blood clotting in response to microbial infection. The Global Sepsis Alliance declared that sepsis is a global emergency with about 6 to 8 million lives lost annually. Patients who survive sepsis often endure long-term cognitive and functional declines.

Current management strategies for sepsis are largely supportive and include early administration of broad-spectrum antibiotics, fluid resuscitation, source control and mechanical ventilation. Despite these strategies, the ICU mortality rate from sepsis remains high (15% to 30%) and risk assessment remains a challenge. Sepsis diagnosis is also a challenge since the clinical features of sepsis closely resemble those of non-infectious systemic inflammatory response syndrome (SIRS). Thus, early recognition of sepsis would improve outcomes.

The identification of highly reliable outcome predictors in sepsis is important to stratify or enroll patients in clinical trials of new anti-sepsis therapies, to monitor a patient's response to treatment, to enhance confidence in end-of-life decision making, and to improve health care resource utilization. Various clinical scoring systems have been developed such as the Acute Physiology and Chronic Health Evaluation (APACHE) II, III, and IV scores, the Multiple Organ Dysfunction Score (MODS) score and the Sequential Organ Failure Assessment (SOFA) score. However, these scores have only a moderate discriminative power with respect to ICU/hospital mortality. Using Receiver Operating Characteristic (ROC) curves, the predictive powers of single measures of these clinical scores, were found to be modest with areas under the curve (AUCs) ranging from 0.6 to 0.7.

It would thus be desirable to develop a risk assessment tool with improved predictive capabilities with respect to risk of death in sepsis patients.

### SUMMARY OF THE INVENTION

It has now been found that the risk of mortality in a septic patient changes over time, and such changes are based on a set of six time-varying biological indicators (TVBIs). These TVBIs include cell-free DNA (cfDNA), protein C, lactate, platelet count, creatinine, and Glasgow Coma Score (GCS), which may collectively be used as indicators to identify septic patients at risk of death.

Thus, in one aspect of the present invention, a method of assessing mortality risk in septic patients, for example patients admitted into the ICU, is provided comprising determining in a biological sample obtained from a patient the level of each of cfDNA, protein C, lactate, platelet count, creatinine, and GCS, and comparing the level of each to a baseline, control or normal level, and providing an assessment of mortality risk, wherein an elevated level of any one of cfDNA, lactate and creatinine or a lowered level of any one of protein C, platelets and GCS is indicative of increased risk of death in the patient.

In another aspect of the invention, a method for determining the probability of dying on a specific day or within a certain time frame (such as within 28 days) is provided comprising the computation from the observed values of the 6 indicators (cfDNA, protein C, lactate, platelet counts, creatinine, and GCS) of a patient in question and the estimated coefficients of the explanatory variables in the CLOGLOG model.

In another aspect of the present invention, a method for monitoring a patient's response to treatment is provided. The method comprises determining in a biological sample obtained from a patient the baseline level of each of cfDNA, protein C, lactate, platelet count, creatinine, and GCS at the onset of treatment, and one or more treatment levels at one or more time points following onset of treatment, comparing the treatment level of each indicator to the baseline level, and providing an assessment of mortality risk, wherein a reduced level of any of cfDNA, lactate and creatinine or an increased level of any of protein C, platelets and GCS indicates that the patient is responding to treatment.

In another aspect of the invention, personalized mortality risk profiles for a patient may be generated based on changing values of the present time-varying biological indicators. The method comprises determining the levels of each of the indicators over time when the patient is septic, and determining the changes in the level of one or more of the indicators that is associated with a decline in the state of the patient and providing a risk profile for the patient which indicates the level of change of the one or more indicators that is indicative of risk of death in the patient.

In another aspect of the invention, a method of detailed ROC analysis is provided for finding the threshold probabilities that can achieve the objectives of (1) maintaining a chosen level of sensitivity, specificity, positive predictive value (PPV), or negative predictive value (NPV), (2) maximizing a weighted sum of these desirable but conflicting measures, and (3) getting the best balance between sensitivity and specificity or between PPV and NPV.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

These and other aspects of the invention are described in the detailed description that follows by reference to the following figures.

### BRIEF DESCRIPTION OF THE FIGURES

FIG. 1 shows a schematic diagram of the risk assessment tool for patients with sepsis.

FIG. 2 shows the temporal patterns of the observed and predicted Daily Hazards of Dying (DHD). The circles represent the observed daily hazards of dying. The dotted dark grey and dotted light grey lines trace the DI-ID predicted by the day 1 specification and the current specification of the CLOGLOG model, respectively. The smooth grey line traces the DHD predicted by the null model, which contains duration and log(duration) as the only two explanatory variables.

FIG. 3 shows the exponential form and the power form for the specification of the dependence of the hazard on the current variable of platelet count in the current specification of the CLOGLOG model. The power form has a greater curvature and a flatter tail

FIG. 4 shows the additive contributions of the time-varying biological indicators to the difference between survivors and non-survivors in the natural log of the hazard of dying. Similar patterns are observed between the Day 1 and Current Specifications of the CLOGLOG Model.

FIG. 5 shows the relative predictive powers and temporal patterns of the TVBIs. Top panel, the relative contributions of the day 1 and change variables of the 6 TVBIs to their combined predictive power in 356 septic patients (difference in the log of hazard of dying between non-survivors and survivors). The sizes of areas are proportional to their shares of their combined predictive power. Bottom panel (A to F), temporal patterns of the daily averages of GCS (A), lactate (B), cfDNA (C), protein C (D), platelet count (E), and creatinine (F). For each TVBI, the septic patients were divided into four quartile groups based on the values of their day 1 variable: best quartile group, second best quartile group, third best quartile group, and worst quartile group. The normal levels in healthy individuals are: 15 for GCS, 0.5-1.0 mmol/L for lactate, 2.2±0.6 μg/ml for cfDNA, 61% to 133% of normal for protein C, 150-400×10⁹/L for platelets, and ≤100 μmol/L for creatinine.

FIG. 6 shows the differences between non-survivors and survivors in the mean contributions to the log of predicted mortality hazards by the time-varying indicators in the current specification of the CLOGLOG (contrast between septic and non-septic patients).

FIG. 7 shows personalized mortality risk profile that highlights the relative contribution of each TVBI to the risk of dying. The profile provides information about how different TVBIs affect the patient's risk of dying on a given day relative to a benchmark representing the best 10th percentile of survivors in terms of the predicted hazard of dying as of the last day. The top panel shows the separate effects of day 1 and change variables of each TVBI. The middle panel shows the net additive effects of the day 1 and change variables for each TVBI. Since hazard ratios (HRs) are easier to interpret than differences in the log of hazard, the latter measures were converted into the former measures. For ease of visualization, the HRs are expressed as “HR-1” as shown in the bottom panel.

FIG. 8 shows personalized mortality risk profile of Patient B that highlights the relative contribution of each TVBI to the risk of dying.

FIG. 9 shows the ROC curves for the derivation and validation groups using the probability of dying in 28 days as the classifier. Black curve: derivation group, N=356, AUC=0.903 (95% CI, 0.864-0.941). Light grey curve with squares: validation group, N=28, without redefining day 1, AUC=0.939 (95% CI, 0.849-1.000). Dark grey curve with circles: validation group, N=28, with day 1 redefined, AUG=0.886 (95% CI, 0.746-1.000).

FIG. 10 shows personalized mortality risk profile that highlights the relative contribution of each TVBI to the risk of dying: generated by the Longitudinal Logit Model.

### DETAILED DESCRIPTION

A method of assessing mortality risk in a septic mammal, for example a patient admitted into the ICU, is provided comprising determining in a biological sample obtained from a patient the level of each of the time-varying biological indicators, cfDNA, protein C, lactate, platelet counts, creatinine, and GCS, and comparing the level of each to a normal or control level, or to a previously determined level, wherein an increase in the level of any of cfDNA, lactate and creatinine as compared to the normal or previously determined level, or a decrease in the level of any of protein C, platelets and GCS as compared to the normal or previously determined level, is indicative of increased risk of death in the mammal.

The term “mammal” includes human and non-human mammals such as a domestic animal (e.g. dog, cat, cow, horse, pig, goat and the like) or a non-domestic animal. A mammal is considered to have sepsis, or to be septic, when body temperature is abnormally higher or lower than normal, heart rate is high, respiratory rate is high and the mammal has a confirmed or probable infection (by an infectious agent such as a virus, bacteria, fungi such as ringworm, nematodes such as parasitic roundworms and pinworms, arthropods such as ticks, mites, fleas, and lice, and other macroparasites such as tapeworms and other helminths). A human is considered to be septic when, along with at least one dysfunctional organ system and confirmed or suspected infection, the patient has at least three of: i) core body temperature is above 100.4° F. (38.3° C.) or below 96.8° F. (36° C.), ii) heart rate is ≥90 beats a minute, iii) respiratory rate is ≥20 breaths a minute or a PaCO₂ (partial pressure of carbon dioxide in arterial blood) is ≤32 mm Hg or the patient requires mechanical ventilation for an acute respiratory process; and iv) a white-cell count of ≥12,000/mm³ or ≤4,000/mm³ or a differential count showing >10 percent immature-eutrophils.

The term “cfDNA”, or “circulating free DNA”, refers to DNA fragments released to the blood plasma, which are generally released by activated neutrophils to aid in killing pathogens. However, the release of excessive amounts of cfDNA can also exert collateral damage to the host by activating blood clotting and inhibiting clot breakdown. The normal level of circulating cfDNA is about 2.2±0.6 μg/ml.

The term “protein C” (also known as vitamin K-dependent protein C preproprotein), is a natural anticoagulant that prevents the accumulation of blood clots in the small vessels of organs. As used herein, protein C encompasses full-length mammalian protein C, including functionally equivalent variants and isoforms thereof, such as human and non-human protein C. Transcript sequences of various forms of full-length protein C are known and readily accessible on sequence databases, such as NCBI, by reference to nucleotide accession nos., e.g. human protein C (NM 000312), mouse protein C (NM_001042767) and canine protein C (NM_001013849.1). Protein C amino acid sequences are also known such as human (NP_000303), mouse (NP_001036232) and canine (NP_001013871.1). Normal levels of protein C are about 61% to 133% of the protein C levels in plasma pooled from healthy volunteers (which is set at 100%). Increased consumption of protein C, i.e. a decrease in the level of protein C from a baseline level, is indicative of sepsis.

The term “lactate” refers to the conjugate base of lactic acid which plays a role in several biochemical processes. The normal level of circulating lactate is about 0.5-1.0 mmol/L. Higher levels of lactate (above a normal or baseline level) are indicative of poor oxygen delivery to organs, presumably due to macro- and/or microcirculatoiy dysfunction, and may reflect tissue hypoperfusion or cellular hypoxia.

The term “platelet”, also called thrombocytes, are a component of blood that function to prevent bleeding from blood vessel injury by initiating blood clotting. Normally, the number of circulating platelets or platelet count is in the range of about 150,000 to 450,000 platelets per microliter of circulating blood. Lower circulating levels of platelets (i.e. below a normal or baseline level) is indicative of sepsis.

Creatinine is a breakdown product of creatine phosphate in muscle, and is usually produced at a fairly constant rate by the body. Normal levels of creatinine is about ≤100 μmol/L in the blood. Elevated levels are indicative of kidney failure.

The term “GCS” as used herein refers to the Glasgow Coma Score as previously described by Marshall J C et al. (Crit Care Med 1995; 23: 1638-52), based on the Glasgow Coma Scale originally described by Teasdale G and Sennett B (Lancet 1974; 2: 81-4). The Glasgow Coma Scale provides a practical method for assessment of impairment of conscious level in response to defined stimuli. It is a neurological scale that provides a reliable and objective way of recording the conscious state of a person for initial as well as subsequent assessment by monitoring eye response, verbal response and motor response. A patient is assessed against the criteria of the scale, and the resulting points give a patient score between 3 (indicating deep unconsciousness) and either 14 or 15 (indicating normal, either based on original or more widely used modified or revised scale, respectively). Thus, the greater the score, the more improved the patient. Neurological dysfunction, i.e. a reduced GCS score, is indicative of sepsis.

The levels of each of the time-varying biological indicators is determined in order to assess mortality risk in a mammal. A suitable biological sample is obtained to measure one or more of the time-varying biological indicators. For example, cell-free DNA, protein C, lactate and creatinine may be measured in biological fluids such as plasma, serum, whole blood, urine, saliva, sweat, tears, and cerebrospinal fluid (CSF).

Cell-free DNA may be measured using DNA extraction techniques (e.g. including removal of cellular debris by centrifugation, and removal of components such as lipids using detergents/surfactants, and removal of protein and RNA using suitable enzymes (proteases and RNase); detection by UV spectrometry (absorbance at 280 nm), DNA dye staining (e.g. with Picogreen, SYBR-green), microfluidics technology (e.g. Picogreen fluorescent dye-labelling), followed by quantitation using an electric current, polymerase chain reaction (PCR) with sequence-specific primers, restriction enzymes and ethidium bromide staining, Slot blot or Southern blotting technology.

Protein C may also be measured in plasma, serum, or whole blood by immunoassay, such as indirect immunoassay, sandwich immunoassay and competitive binding assay, or microfluidics technology using a monoclonal or polyclonal antibody against human protein C. As one of skill in the art would know, antibodies specific for protein C are commercially available (e.g. from Thermofisher, Abeam, Novus Biologicals). Alternatively, antibodies for this purpose may be raised by injecting a non-human host animal, e.g. a mouse or rabbit, with antigen (protein C or immunogenic fragment thereof), and then isolating antibody from a biological sample taken from the host animal.

A preferred immunoassay for use to determine expression levels of target protein in a sample is an ELISA (Enzyme Linked ImmunoSorbent Assay) or Enzyme ImmunoAssay (EIA). To determine the level or concentration of the target protein using ELISA, the target protein to be analyzed is generally immobilized, for example, on a solid adherent support, such as a mierotiter plate, polystyrene beads, nitrocellulose, cellulose acetate, glass fibers and other suitable porous polymers, which is pretreated with an appropriate ligand for the target, which is then complexed with a specific reactant or ligand such as an antibody which is itself linked (either before or following formation of the complex) to an indicator, such as an enzyme. Detection may then be accomplished by incubating this enzyme-complex with a substrate for the enzyme that yields a detectable product. The indicator may be linked directly to the reactant (e.g. antibody) or may be linked via another entity, such as a secondary antibody that recognizes the first or primary antibody. Alternatively, the linker may be a protein such as streptavidin if the primary antibody is biotin-labeled. Examples of suitable enzymes for use as an indicator include, but are not limited to, horseradish peroxidase (HRP), alkaline phosphatase (AP), β-galactosidase, acetylcholinesterase and catalase. A large selection of substrates is available for performing the ELISA with these indicator enzymes. As one of skill in the art will appreciate, the substrate will vary with the enzyme utilized. Useful substrates also depend on the level of detection required and the detection instrumentation used, e.g. spectrophotometer, fluorometer or luminometer. Substrates for HRP include 3,3′,5,5′-Tetramethylbenzidine (TMB), 3,3′-Diaminobenzidine (DAB) and 2,2′-azino-bis(3-ethylbenzothiazoline-6-sulphonic acid) (ABTS). Substrates for AP include para-Nitrophenylphosphates. Substrates for β-galactosidase include β-galactosides; the substrate for acetylcholinesterase is acetylcholine, and the substrate for catalase is hydrogen peroxide.

Isoelectric focusing may also be used to measure protein C whereby protein C is separated and quantified according to its isoelectric point within a continuous pH gradient. Protein C can also be quantified using a chromogenic assay in which protein C in the plasma is activated (e.g. by addition of an activator such as the snake venom, Protac) and the level of activated protein C (APC) may be measured by determining change in optical density in the presence of a chromogenic substrate specific to APC (such as S-2366) which results in a colour change and comparing to a standard APC curve. A functional clotting-based assay such as the Activated Partial Thromboplastin Time (APTT) assay may also be used to measure Protein C. Briefly, plasma is incubated at 37° C. with phospholipids, a contact activator (e.g. kaolin), and a protein C activator (e.g. the snake venom, Protac). After a few minutes of incubation, CaCl₂ is added to initiate clotting. The time required to clot is recorded, and the protein C concentration is determined from a reference curve of plasma containing different concentrations of protein C.

Lactate and creatinine may be measured in a plasma, serum, or whole blood sample using an enzymatic assay to generate a product that may be detected colorimetrically or fluorometrically by reaction with a selective probe. To measure lactate in a sample, lactate dehydrogenase or lactate oxidase assays may be used. To measure creatinine in a sample, a creatininase assay in which creatine from creatinine is converted to sarcosine which is oxidized with sarcosine oxidase to produce a product which reacts with a probe for colorimetric or fluorescent quantitation. Lactate can be also measured using electrode methods, such as blood gas analyzers. Lactate can be measured in CSF (cerebral spinal fluid) and other body fluids while creatinine can also be measured in urine. Colorimetric assays may also be used to measure creatinine, for example, using the Jaffe method in which creatinine is reacted with picric acid to yield a detectable product.

Platelet count is measured in a blood sample obtained from a patient, either from a vein, or finger or heel smear. A hematology analyzer (cell counter) or POC devices for complete blood count (CBC) testing may be used.

The GCS comprises three tests performed at bedside: eye response (rated 1 to 4), verbal response (rated 1 to 5), and motor response (rated 1 to 6). Both the three individual element values as well as their sum are considered important. The following provides a summary of the grading for eye response: does not open eyes (1), opens eyes in response to pressure (2), opens eyes in response to voice (3), and opens eyes spontaneously (4); verbal response: makes no sounds (1); makes sounds (2), words (3), confused disoriented (4), oriented, converses normally (5); and motor response: makes no movements (1), extension to painful stimuli (decerebrate response) (2), abnormal flexion to painful stimuli (decorticate response) (3), flexion/withdrawal to painful stimuli (4), localizes to painful stimuli (5) and obeys commands (6).

Once the levels of each time-varying biological indicator is determined, they may be used to assess mortality risk in a septic mammal. The determined level of each indicator is compared to a normal or control level of the indicator, i.e. a level of the indicator in corresponding healthy individuals. An increase of at least about 1.5-fold in the level of any of cfDNA, lactate and creatinine, or a decrease in the level of protein C to ≤65% of normal levels, of platelets to ≤200×10⁹/L or a 10% decrease/day, or a decrease of GCS to ≤12, is indicative of increased risk of death in the mammal, for example, within 28 days.

The levels of the indicators may alternatively be compared to a previously determined level in a septic mammal being assessed for risk of death. This will provide the change in the level of a given indicator in the septic mammal. An increase of at least about 1,5-fold in the level of any of cfDNA, lactate and creatinine, or a decrease in the level of protein C and platelets by about 1.5-fold from normal, or a decrease in GCS, is indicative of increased risk of death in a mammal.

In an assessment of a mammal, it will be appreciated that the determined level of each indicator may be compared to a combination of normal/control indicator levels and previously determined indicator levels. For example, in one embodiment, levels of cfDNA, protein C, platelets and GCS may be compared to a control or normal level (e.g. the initial level of these indicators is utilized for the assessment), while determined levels of lactate and creatinine are compared to a previously determined level of these indicators in the septic mammal (i.e. the change in the level of these indicators is utilized in the assessment).

It will also be appreciated that two or more indicator levels may be evaluated to determine risk of death in a septic patient. The indicators utilized for the assessment may vary from patient to patient within the group of cfDNA, protein C, platelets, lactate, creatinine and GCS.

The indicator levels may also be determined at a plurality of time-points to obtain time-varying indicator values in the risk assessment.

In another aspect of the invention, a method for determining the probability of dying on a specific day or within a certain time frame (such as within 28 days) is provided comprising determining in a biological sample the level of the 6 biological indicators (cfDNA, protein C, lactate, platelet counts, creatinine, and GCS) in comparison to control or previously determined levels of the indicators. The probability of dying is then determined based on a complementary log-log analysis of the levels of one or more of the time-varying indicators as described in the examples.

In another aspect of the invention, personalized mortality risk profiles for a patient may be generated based on changing values of the present time-varying biological indicators. The method comprises determining the levels of each of the indicators over time when the patient is septic, and determining the changes in the level of one or more of the indicators as compared to control levels (or benchmark levels) that is associated with a decline in the state of the patient and providing a risk profile for the patient which indicates the level of change of the one or more indicators that is indicative of risk of death in the patient. A longitudinal logit (L-Logit) model or complementary log-log analysis of the change in indicator levels is conducted as described in the examples. An increased risk of mortality is determined when the profile indicates an increase in the level of any one of cfDNA, lactate and creatinine, or decrease in the level of any one of protein C, platelets and GCS, or an increased probability of death based on the complementary log-log analysis. This method is useful to provide insights into patient-specific pathophysiology, to develop a treatment protocol for a patient, and for prognostic and predictive enrichment.

In another aspect of the present invention, a method for monitoring a patient's response to treatment is provided. The method comprises determining in a biological sample obtained from a patient the level of each of cfDNA, protein C, lactate, platelet count, creatinine, and GCS a baseline level of the indicators at the onset of treatment and one or more treatment levels at one or more time points following onset of treatment, comparing the level of each indicator to the baseline level, wherein a reduced level of any cfDNA, lactate and creatinine or an increased level of any of protein C, platelets and GCS, i.e. a return of one or more of the indicators to normal levels, indicates that the patient is responding to treatment. Such a method is useful to confirm suitability of a selected treatment, and further, to stratify/enroll patients for clinical trials of new anti-sepsis therapies.

The foregoing methods are beneficial to ascertain the appropriate type and level of care for a septic patent. In particular, the methods are useful to determine appropriate treatment for a given patient, i.e. one or a combination of reducing cfDNAs, lactate and/or creatinine levels, and/or increasing protein C, platelet levels, and/or GCS. For example, recombinant ART-123 is a molecule that has been determined to boost protein C levels. Where cfDNA levels are determined to be increased, treatments to inhibit blood clotting may be used, for example, anticoagulants such as heparin, warfarin (Coumadin), Rivaroxaban, Dabigatran, Apixaban, or antiplatelet drugs such as aspirin.

Other treatments for a septic patient may be administered to boost the immune system. These may include treatment with mesenchymal stem cells, herbal remedies such as Echinacea and ginseng, probiotics, and diet enhanced with immune boosting nutrients, vitamins and minerals (e.g. fruits and vegetables, fish (omega-3), shellfish (selenium), zinc-containing foods (beef), garlic (allicin), etc.

The present methods are also beneficial when considering end-of-life decision making, to enhance confidence in such decisions, and to improve health care resource utilization.

Terms of degree such as “about” or “approximately” as used herein refer to a reasonable amount of deviation from a stated quantity which does not significantly change the end result such as +/−5-10%.

Embodiments of the present invention are described in the following specific examples which are not to be construed as limiting.

### EXAMPLES

To link the mortality risks of patients to the TVBIs, a novel approach for longitudinal analysis, termed the complementary log-log (CLOGLOG) model, was chosen. It has also been found that personalized mortality risk profiles can be generated which highlight the relative contribution of each TVBI for mortality risk.

The TVBIs that are missing in the APACHE II/III/IV, MODS, and SOFA scores but have been shown to have prognostic utility in septic patients include plasma concentrations of lactate, cfDNA, and protein C.

A multi-centre study of 392 septic patients was performed to determine the applicability of this assessment tool for determining the hazards of dying during the patients' stay in ICU/hospital up to 28 days. The assessment tool was also tested on 328 non-septic ICU patients to determine whether the pattern of the effects of the indicators is unique to septic patients. Blood samples were collected at baseline (within 24 hours of meeting the inclusion criteria for sepsis), then daily for the first week, followed by once a week for the duration of the patients' stay in the ICU, cfDNA, protein C, lactate, platelets, and creatinine levels may be determined from patient blood samples, whereas the GCS is a neurological scale that measures eye, verbal, and motor responses at the bedside.

A complementary log-log (CLOGLOG) model that followed the daily life of each patient until death in ICU/hospital, discharge, or 28 days since admission to predict the mortality risk over time and generate personalized mortality risk profiles that highlight the relative contribution of each TVBI for mortality risk was used. Each TVBI was represented by three analytical variables: day 1 variable, current variable, and change variable. The first two variables were alternatives for quantifying the level effect, whereas the third variable was for quantifying the change effect. The model using the combination of day 1 and change variables of each indicator is called the “day 1 specification”, whereas the model using the combination of current and change variables is called the “current specification”. The two specifications are complementary in yielding important biological insights. The combination of day 1 and change variables achieved a predictive power (AUC=0.90 (95% CI, 0.86-0.94)) that is similar to the combination of current and change variables. In both specifications, the assessment was done in the context of two preconditions (chronic lung disease and previous brain injury), age, and duration of stay.

The day 1 variables of a subset of the 6 indicators, namely protein C, lactate, and creatinine, was also used to distinguish septic patients from non-septic patients via a binomial logit model, resulting in AUC=0.67 (CI: 0.63 to 0.71). In general, patients with lower protein C, lower lactate, and higher creatinine were more likely to be septic patients.

### Assessment Methods

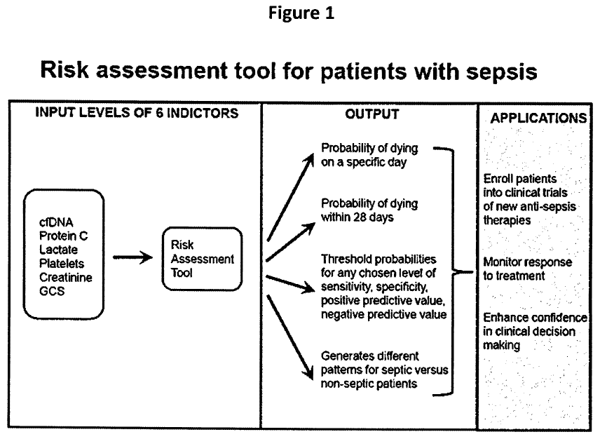

FIG. 1 shows a schematic diagram of the application of the present method. By inputting measured values from the 6 indicators obtained by assessment of patient blood samples (cfDNA, protein C, lactate, platelets, creatinine) or neurological scores (GCS), this risk assessment tool provides several valuable outputs including: the probability of dying on a specific day; the probability of dying within 28 days; the threshold probabilities for any chosen level of sensitivity, specificity, PPV, NPV; which generates different patterns for septic versus non-septic patients. This information can be used to enroll or stratify patients into clinical trials (for example, clinical trials of new anti-sepsis therapies), monitor response to treatment, and enhance confidence in clinical decision making.

392 patients with sepsis or septic shock and 361 non-septic patients were recruited from nine tertiary hospital ICUs across Canada between November 2010 and January 2013 (the DYNAMICS Study, ClinicalTrials.gov Identifier: NCT01355042). The study was approved by the Research Ethics Boards of all participating centers. Written informed consent was obtained from the patient or substitute decision-maker prior to enrolment into the study. When a priori consent was not feasible, a deferred consent approach was used. All septic events were adjudicated by at least 2 experienced ICU physicians. Adjudications were also performed in the non-septic patients to identify those patients who became septic during the course of their stay in the ICU. In total, 33 out of 361 non-septic patients developed sepsis in the ICU. These 33 patients were removed from the analysis of the non-septic patients, so the number of non-septic patients was reduced to 328.

The inclusion criteria for sepsis were a modification of those defined by Bernard et al. (N Engl J Med 2001; 344: 699-709). Patients were eligible for inclusion into the septic group of this study if they had a confirmed or suspected infection on the basis of clinical data at the time of screening, at least one dysfunctional organ system, 3 or more signs of systemic inflammatory response syndrome (SIRS), and were expected to remain in the ICU for ≥72 hours. The presence of organ dysfunction are: (1) SBP≤90 mm Hg or MAP≤70 mm Hg or SBP≤40 mm Hg for at least 1 hour despite fluid resuscitation, adequate intravascular volume status, or use of vasopressor in an attempt to maintain systolic BP≥90 or MAP≥70 mm Hg; (2) P/F Ratio ≤250 in the presence of other dysfunctional organs or systems, or ≤200 if the lung is the only dysfunctional organ; (3) acute rise in creatinine >171 mM or urine output <0.5 ml/kg body weight for 1 hour despite adequate fluid resuscitation; (4) unexplained metabolic acidosis (pH≤7.30 or base deficit ≥5 with lactate >1.5 times the upper limit of normal; and (5) platelet count <50,000 or a 50% drop over the 3 days prior to ICU admission. The inclusion criteria for septic shock are the same as those for sepsis except that the patient must be on vasopressors within the previous 24 hours. Patients were excluded if they were <18 years old, were pregnant or breastfeeding, or were receiving palliative care only.

To meet the inclusion criteria for non-sepsis, patients must have been classified as: (A) patients with multiple trauma with an episode of shock who were expected to remain in the ICU for ≥72 hours (shock must have been present within the previous 24 hours and may have resolved at the time of enrolment). Shock is defined as SBP≤90 or MAP≤70 mm Hg or SBP<40 from baseline, or lactate >1.5 times the upper limit of normal, or other evidence of acute organ dysfunction; or (B) critically ill patients who were expected to remain in the ICU for ≥72 hours (e.g. intracerebral hemorrhage, subarachnoid hemorrhage, subdural hemorrhage); or (C) patients with non-septic shock (e.g. cardiogenic shock, hypovolemia, heat shock, burns requiring mechanical ventilation, pulmonary embolism, abdominal aortic aneurysm) who were expected to remain in the ICU for ≥72 hours (shock must have been present within the previous 24 hours and may have resolved at the time of enrolment).

Baseline characteristics include demographic information, organ function, pre-existing chronic conditions, sites of infection, types of infection, APACHE II score, and use of vasopressor/inotropes. Daily data included microbiologic culture results, organ function, hematologic and other laboratory tests, and type and quantity of resuscitation fluid.

In the septic patients, 88% of the admissions were medical, 94% of the patients required mechanical ventilation, and 67% required vasopressors or inotropes. The main site of infection was the lung accounting for 42% of the patients. The 28-day mortality rate was 23.5%.

In the non-septic patients, 75% of the admissions were medical, 90% of the patients required mechanical ventilation, and 43% required vasopressors or inotropes. The 28-day mortality rate was 18.3%.

The patient blood samples were collected within 24 hours of meeting the inclusion criteria for severe sepsis. Blood samples and clinical data were obtained at baseline, then daily for the first week, followed by once a week for the duration of the patients' stay in the ICU. The blood was processed within two hours of blood collection. Briefly, blood (10 ml each) was collected from existing arterial or venous lines (or by venipuncture with a 20-gauge needle) into Becton Dickinson buffered sodium citrate vacutainer tubes (0.105M trisodium citrate). The blood was centrifuged at 1,500×g for 10 min at 20° C., and the plasma was stored as 200 uL aliquots at −80° C. and thawed at the time of assays.

Plasma samples were obtained from 33 healthy adult volunteers who were not receiving any medication at the time of blood sampling. No attempt to match cases and controls was made.

Levels of the six indicators were determined as follows:

Lactate and creatinine were measured via enzymatic digestion using commercially available assays, namely, the Lactic Acid assay (lactic acid conversion to pyruvate and hydrogen peroxide by lactate oxidase) run on the ARCHITECT cSystem by Abbott, and the Creatinine assay (Kinetic Alkaline Picrate: creatinine reaction with picrate to form a creatinine-picrate complex at an alkaline pH) run on Abbott's ARCHITECT c Systems and AEROSET System.

A hematology analyzer (cell counter) was used to measure platelet count.

In this study, cfDNA was isolated from 200 μL of plasma using the QIAamp DNA Blood Mini Kit (Qiagen, Valencia, Calif.). The concentration of the DNA was measured by UV absorbance at 260 nm using a spectrophotometer (BioPhotometer Plus spectrophotometer, Eppendorf, Mississauga, ON). The purity of the DNA was confirmed by determining the OD₂₆₀/OD₂₈₀ ratio.

Plasma levels of protein C antigen were quantified by an enzyme immunoassay (Affinity Biologicals Inc., Ancaster, ON).

The GCS was measured at the bedside.

Statistical analyses: The mortality risks within 28 days since admission were assessed to formulate a multivariate model in the following way. Using a day as the unit of time, a longitudinal approach was used that follows the daily life of each patient until (1) death in ICU or hospital, or (2) discharge from hospital, or (3) the date of censoring on day 28 since ICU admission. Let Hᵢₜ be the daily hazard of dying of the ith patient on day t. The model linking Hᵢₜ to the explanatory variables is:

Hᵢₜ=e^{β}^{₀}^{+β′X}^{ᵢₜ} for i=1,2, . . . n and t=1,2, . . . Tᵢ (1)

where 13₀ is an unknown intercept, β′ is a row vector of unknown coefficients; Xᵢₜ is a column vector of the explanatory variables that reflect the relevant information of the ith patient up to day t; n is the number of patients in the sample; and Tᵢ is the day when the ith patient died in ICU/hospital, was discharged, or was censored (day 28). A discharge is defined as the transfer of a live patient from the ICU or hospital to home or other institution where the information on mortality status was no longer collected. Since each live patient is censored on day 28, both the day of death and the day of discharge are ≤28. Implicit in this formulation is the simplifying assumption that the hazard remains constant through all time points within each day. For simplicity, t is called the “current day” and Tᵢ is called the “last day” of the ith patient since admission.

For each of the six time-varying biological indicators (TVBIs), the following three analytical variables were defined: (I) the day 1 variable, which assumes the same day 1 value of the indicator for all t; (2) the current variable, which in its simple form assumes the observed (directly observed or imputed) value of the indicator on day t; and (3) the change variable, which is defined as the day 1 variable minus the current variable. Any value of a current variable that is not directly observed was imputed as follows. If the day in question is preceded by at least one day with directly observed value and is followed by at least one day with directly observed value, then it is linearly interpolated from the two closest observed values. Otherwise, it is set to be equal to the nearest observed value.

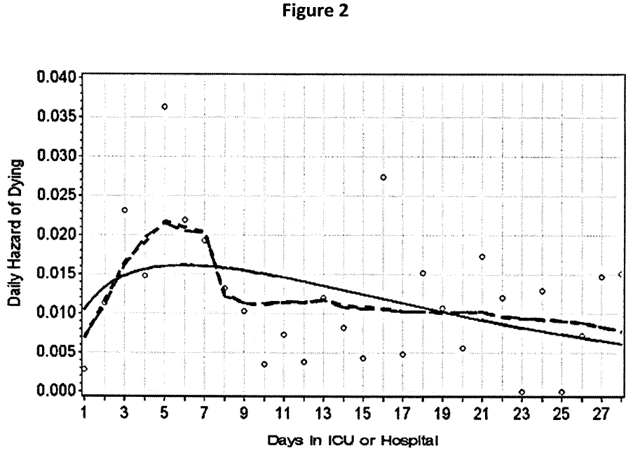

To reduce the risk of making misleading inferences from observational data, the model includes the following explanatory variables for representing the relevant context in both specifications of the CLOGLOG model. First, two dummy variables were used to represent the presence or absence of the preconditions of chronic lung disease and previous brain injury. Second, age was used to represent the demographic background. Third, the duration of stay and its natural log transformation were used to represent the temporal pattern of the hazard that resulted from a balance of several processes (e.g. the death process that tended to remove relatively sick patients from the sample, and the discharge process that tended to remove relatively healthy patients). Duration and log(duration) were used as two of the explanatory variables (1) to help capture the temporal pattern of the hazard of dying and (2) to prevent selection biases from resulting in misleading findings. Mathematically, this specification of the time function expresses the dependence of hazard on duration as a product of an exponential function and a power function. It has the advantage of being highly flexible in reflecting the temporal pattern in the data. Using duration and log(duration) as the only two explanatory variables in the CLOGLOG model, the estimated function Ĥₜ=e^{-4.4855-0.0755t}(t^{0.4548}), where t is the duration of stay and Ĥₜ is the estimated hazard of dying on day t was obtained. This function is represented by the smooth grey curve in FIG. 2. The curve peaked in the later part of the first week and then declined. Some TVBIs may have both level and change effects on the hazard of dying. Either the day 1 variable or the current variable is used in the model for estimating the level effect, whereas the change variable is included in the model so that its coefficient can represent the change effect. For convenience, the day 1 and current variables are called the level variables. To obtain complementary insights on determining whether it is better to use day 1 variables or current variables to quantify the level effects of TVBIs, two specifications of the CLOGLOG model were used: a “day 1 specification” that combined day 1 variables with change variables, and a “current specification” that combined current variables with change variables. To estimate the unknown coefficients of each specification of the CLOGLOG model, the maximum likelihood method was used in the following way. The daily hazard of dying Hᵢₜ was first translated into the daily probability of dying, Pᵢₜ. Since Hᵢₜ is assumed to be constant through all time points on day t, let Pᵢₜ be the ith patient's probability of dying on day t, conditional on the survival up to the beginning of day t. Eq. (1) then implies:

Pᵢₜ=1−e^{-H}^{ᵢₜ}=1e^{-eβ}^{_{O}}^{+β}^{¹}^{X}^{ᵢₜ} (2)

Let Yᵢₜ be a dummy variable that assumes the value of 1 if the ith person died on day t. The unknown coefficients are then estimated by maximizing the following log-likelihood function:

$$L ( {β}_{0} , β ) = {\sum }_{i = 1}^{n} {\sum }_{t = 1}^{{T}_{i}} [ {Y}_{it} ln ( {P}_{it} ) + ( 1 - {Y}_{it} ) ln ( 1 - {P}_{it} ) ] \qquad ( 3 )$$

To carry out the estimation, the Logistic procedure of SAS with the option of LINK=CLOGLOG was used (Allison, P. D. Survival Analysis Using SAS, 2010, pp. 240-247. Note that Eq. (2) can be rewritten as:

ln(−ln(1−Pᵢₜ))=β₀+Xᵢₜ (4)

Since the left-hand-side is called a complementary log-log function of Pᵢₜ, this model is called a complementary log-log (CLOGLOG) model. The CLOLOG model is similar to but more versatile in yielding longitudinal insights than the Cox proportional hazards model, as the CLOGLOG model is free from the restriction of the proportional hazards assumption and is capable of generating the predicted probability of dying on any day or in any time interval. The similarity is in the expression of the dependent variable (the daily hazard of dying) as an exponential function of explanatory variables. The versatility derives from the ease of including a large number of time-varying variables and the replacement of the maximum partial-likelihood method by the maximum likelihood method for estimation. The maximum partial-likelihood method does not represent the removed time-dependent part of the model by an unknown constant and hence does not generate an estimated intercept, which is needed for computing the predicted hazard that is to be translated into easily interpretable probabilities. Instead of starting with Eq. (4), in which the model may be considered as a discrete-time model, formulation of the model with Eq. (1) is a continuous-time model. It is easier to see from Eq. (1) that the exponential transformation of the kth element of β is the hazard ratio for the kth explanatory variable.

For several reasons, the CLOGLOG model is preferred over the conventional logistic model of the form:

$${P}_{i} = \frac{{e}^{{e}^{{β}_{0} + {β}^{′} {X}_{i}}}}{1 + {e}^{{e}^{{β}_{0} + {β}^{′} {X}_{i}}}} \qquad ( 5 )$$

where Pᵢ is the ith patient's probability of dying in 28 days. First, for these data, the latter involves the unrealistic assumption that none of the discharged patients died, whereas the former does not. Second, the former can reveal the temporal pattern of the risk of dying, whereas the latter cannot. Third, for the assessment of treatment effect, the latter often yields spurious findings, whereas the former does not. For example, CLOGLOG model demonstrates that clinicians had a strong tendency to apply vasopressors to sicker patients so that the dummy variable representing the application of vasopressors had a very high hazard ratio (HR) of 5.6, and that the beneficial effect of vasopressors in reducing mortality risk became statistically significant after the initial week, causing the HR to decrease sharply to 1.3. In contrast, the conventional logistic model yielded an odds ratio of 2.4 for the dummy variable, incorrectly suggesting that the application of vasopressors resulted in worse mortality outcomes. Note that in both models, age and the preconditions of chronic lung disease and previous brain injury were used as contextual variables, and that in the CLOGLOG model, duration and log(duration) were used as additional contextual variables to represent the overall temporal pattern of the hazard.

A more useful version of the logistic model is of the form:

$${P}_{it} = \frac{{e}^{{e}^{{β}_{0} + {β}^{′} {X}_{it}}}}{1 + {e}^{{e}^{{β}_{0} + {β}^{′} {X}_{it}}}} \qquad ( 6 )$$

where Pᵢₜ is the ith patient's probability of dying on day t, conditional on surviving to the beginning of day t. If the explanatory variables are skillfully specified, and if the additive components of the predicted “logit” (log of odds) are also skillfully used, then the qualitative insights obtained from it will be very similar to those obtained by the CLOGLOG model and can be considered its discrete-time analogue. Due to its usefulness for longitudinal data analysis, the name Longitudinal Logit Model to this version, which is analogous to the CLOGLOG model, is offered.

The model was applied to the septic and the non-septic groups separately. The observations in the input data file for each group are the daily records of all patients with observed values for the explanatory variables. The number of observations for each patient who died in ICU or hospital is equal to the number of days from admission to the day of death, whereas the number of observations for each patient who was discharged is equal to the number of days from admission to the day of discharge. Each of the censored patients contributes 28 observations. The input data matrix has a simple structure. Each row represents a person-day, in which the information of all explanatory variables is used to enhance the likelihood of the value of the outcome variable (Yᵢₜ). The original data file for all 392 septic patients contained 7,298 observations (rows).

Since the Logistic procedure of SAS does not compute the 95% confidence interval for AUC (the area under the curve showing the relationship between sensitivity and 1-specificity in the ROC analysis), a SAS module to carry out the computation was written. In this module, the standard deviation of the AUC was computed according to the algorithm developed by Hanley and McNeil (Radiology. 1982; 143:29-36). To better reflect the effects of the sample size and the number of unknown coefficients on the width of the confidence interval, the critical value for constructing the confidence interval from a t-distribution was taken rather than the standard normal distribution.

Since the daily probability of dying considered herein is a conditional probability, the probability of dying in 28 days should not be computed by adding up 28 daily probabilities of dying. The probability of dying in 28 days implied by the daily hazard of dying H is 1−e^{-28H}.

For the prognosis of individual patients, the present method can generate the threshold probabilities (1) for any chosen levels of sensitivity, specificity, PPV, and NPV, (2) for the best weighted sum of these desirable but conflicting measures, and (3) for the best balance between sensitivity and specificity or between PPV and NPV. This capability originates from the computer algorithm that generates the set of the predicted probabilities of dying within 28 days (or any reasonable duration) for all septic patients, from which a detailed list of threshold probabilities was used to compute the values of these four measures.

### Example 1—Contrasting Septic and Non-Septic Patients and Generating Predicted Hazard and Probabilities of Dying

From the records of 355 septic patients and 288 non-septic patients with non-missing values, the input data sets of the two groups of patients for the CLOGLOG model has 6,712 and 4,950 observations (person-days), respectively. Table 1 shows the estimated results of the day 1 specification of the CLOGLOG model (Panel A for septic patients and Panel B for non-septic patients).

TABLE 1

| | | Wald | | | | | Wald | | | |
|---|---|---|---|---|---|---|---|---|---|---|
| Explanatory | | Chi- | | Hazard | 1/ | | Chi- | | Hazard | 1/ |
| Variable | Coefficient | Square | p-value | Ratio | HR | Coefficient | Square | p-value | Ratio | HR |
| | (A) Septic Patients | | | | | (B) Non-septic Patients | | | | |
| Intercept | −5.4483 | 49.3 | <.0001 | | | −6.9531 | 49.8 | <.0001 | | |
| 1. LEVEL | | | | | | | | | | |
| cfDNA | 0.2120 | 30.4 | <.0001 | 1.69 | — | 0.2037 | 22.1 | <.0001 | 1.97 | — |
| Protein C | −0.01130 | 6.9 | 0.0086 | 0.657 | 1.52 | — | — | — | — | — |
| Platelets | −0.00453 | 11.4 | 0.0007 | 0.576 | 1.74 | — | — | — | — | — |
| Creatinine | — | — | — | — | — | 0.00349 | 15.8 | <.0001 | 1.59 | — |
| GCS | −0.1231 | 13.0 | 0.0003 | 0.576 | 1.74 | −0.3669 | 54.2 | <.0001 | 0.183 | 5.46 |
| Lactate | 0.0688 | 3.9 | 0.0476 | 1.25 | — | 0.1318 | 8.6 | 0.0033 | 1.64 | — |
| 2. CHANGE | | | | | | | | | | |
| cfDNA | 0.2337 | 11.9 | 0.0006 | 1.79 | — | 0.2475 | 16.7 | <.0001 | 1.79 | — |
| Protein C | −0.00797 | 3.3 | 0.0703 | 0.743 | 1.35 | — | — | — | — | — |
| Platelets | −0.00511 | 10.9 | 0.0009 | 0.536 | 1.86 | — | — | — | — | — |
| Creatinine | 0.00231 | 5.1 | 0.0236 | 1.39 | — | — | — | — | — | — |
| GCS | −0.1412 | 24.2 | <.0001 | 0.531 | 1.88 | −0.3059 | 61.7 | <.0001 | 0.243 | 4.11 |
| Lactate | 0.1543 | 22.2 | <.0001 | 1.65 | — | 0.1867 | 9.0 | 0.0027 | 2.02 | — |
| 3. CONTEXT | | | | | | | | | | |
| Chronic Lung | 0.7540 | 8.7 | 0.0032 | 2.13 | — | — | — | — | — | — |
| Disease | | | | | | | | | | |
| Brain Injury | 1.4006 | 7.6 | 0.0060 | 4.06 | — | — | — | — | — | — |
| Age | 0.0165 | 3.7 | 0.0556 | 1.29 | — | 0.0248 | 6.1 | 0.0134 | 1.50 | — |
| Duration of | −0.0597 | 2.1 | 0.1488 | | | −0.1854 | 9.2 | 0.0024 | | |
| Stay | | | | | | | | | | |
| Log(Duration | 1.0301 | 7.3 | 0.0070 | — | | 2.1371 | 16.7 | <.0001 | — | |
| of Stay) | | | | | | | | | | |

| | | |
|---|---|---|
| AUC | 0.891 (CI: 0.850-0.932) | 0.936 (CI: 0.891-0.982) |

For both groups of patients, the signs of the estimated coefficients of the variables representing all 6 indicators turned out to be physiologically sensible (i.e. positive for cfDNA, lactate, and creatinine, and negative for protein C, platelets, and GCS). With respect to the temporal pattern, the signs of the estimated coefficients of “Log(Duration of Stay)” and “Duration of Stay” turned out to be positive and negative, respectively, implying that the hazard of dying increased first and then declined. It is likely that the increase during the first few days reflected the selection bias resulting from removing patients who were expected to die within the first 72 hours. The decrease partly reflects the cumulative benefits of the treatments and care received by the patients in IUC and hospital. The area under the curve in ROC analysis was 0.891 (CI: 0.850 to 0.932) for septic patients and 0.936 (CI: 0.891-0.982) for non-septic patients. Note that the values of hazard ratio, which can be used to assess the relative importance of the explanatory variables, are based on the assumption that all time-varying indicators are comparable after being divided by their respective standard deviations. Also note that protein C and platelets did not have significant effects for non-septic patients.

Table 2 shows the estimated results of the current specification of the CLOGLOG model (Panel A for septic patients and panel B for non-septic patients). The main difference from the results of the day 1 specification of the model was the reduction in the number of change variables with significant coefficients for both septic and non-septic patients. This difference suggests that the current variables retained most of the predictive powers of not only the day 1 variables but also the change variables. In other words, using the most up-to-date information of the 6 indicators largely removed the need to pay attention to the changes from day 1. For septic patients, only the change variables of GCS and creatinine remained in the model. For non-septic patients, none of the change variables remained. The area under the curve in ROC analysis was 0.886 (CI: 0.844 to 0.929) for septic patients and 0.940 (CI: 0.896-0.984) for non-septic patients.

TABLE 2

| | | Wald | | | | | Wald | | | |
|---|---|---|---|---|---|---|---|---|---|---|
| Explanatory | | Chi- | | Hazard | 1/ | | Chi- | | Hazard | 1/ |
| Variable | Coefficient | Square | p-value | Ratio | HR | Coefficient | Square | p-value | Ratio | HR |
| | (A) Septic Patients | | | | | (B) Non-septic Patients | | | | |
| Intercept | −5.4655 | 50.7 | <.0001 | | | −5.6245 | 56.5 | <.0001 | | |
| 1. LEVEL | | | | | | | | | | |
| cfDNA | 0.2098 | 32.0 | <.0001 | 1.69 | — | 0.2051 | 28.1 | <.0001 | 1.98 | — |
| Protein C | −0.00978 | 7.3 | 0.0070 | 0.695 | 1.44 | — | — | — | — | — |
| Platelets | −0.00472 | 14.3 | 0.0002 | 0.562 | 1.78 | — | — | — | — | — |
| Creatinine | — | — | — | — | — | 0.00211 | 3.9 | 0.04960 | 1.33 | — |
| GCS | −0.1371 | 24.6 | <.0001 | 0.541 | 1.85 | −0.2986 | 63.0 | <.0001 | 0.251 | 3.98 |
| Lactate | 0.0707 | 4.2 | 0.0403 | 1.26 | — | 0.1142 | 6.4 | 0.0115 | 1.54 | — |
| 2. CHANGE | | | | | | | | | | |
| cfDNA | — | — | — | — | — | — | — | — | — | — |
| Protein C | — | — | — | — | — | −0.00972 | 4.7 | 0.02960 | — | — |
| Platelets | — | — | — | — | — | — | — | — | — | — |
| Creatinine | 0.00219 | 4.8 | 0.0288 | 1.37 | — | — | — | — | — | — |
| GCS | — | — | — | — | — | — | — | — | — | — |
| Lactate | 0.0867 | 5.0 | 0.0254 | 1.32 | — | — | — | — | — | — |
| 3. CONTEXT | | | | | | | | | | |
| Chronic Lung | 0.7839 | 9.6 | 0.0019 | 2.19 | — | 0.7571 | 4.9 | 0.0262 | 2.13 | — |
| Disease | | | | | | | | | | |
| Brain Injury | 1.3733 | 7.5 | 0.0061 | 3.95 | — | — | — | — | — | — |
| Age | 0.0172 | 4.2 | 0.0393 | 1.30 | — | — | — | — | — | — |
| Duration of | −0.0608 | 2.2 | 0.1374 | | | −0.1825 | 9.0 | 0.0027 | | |
| Stay | | | | | | | | | | |
| Log(Duration | 1.0503 | 7.7 | 0.0054 | — | | 2.0239 | 15.3 | <.0001 | — | |
| of Stay) | | | | | | | | | | |

| | | |
|---|---|---|
| AUC | 0.886 (CI: 0.844-0.929) | 0.940 (CI: 0.896-0.984) |

As shown in Table 3, this risk assessment tool calculates the mortality hazard and probabilities of dying for a septic patient who died on day 11 (based on the estimated coefficients of the current specification of the CLOGLOG model for septic patients). For this patient, the predicated probability of dying on day 11 and within 28 days is 12% and 97%, respectively.

TABLE 3

| | Estimated | Values of Explanatory | Additive Terms with |
|---|---|---|---|
| | Coefficients | Variables | a Common Unit |
| Explanatory Variables | Vector “B” | Vector “X” | Terms in B′X |
| Intercept | −5.4655 | 1 | −5.466 |
| DNA_CURRENT | 0.2098 | 9.6 | 2.014 |
| PROTC_CURRENT | −0.00978 | 30 | −0.293 |
| Platelets_CURRENT | −0.00472 | 27 | −0.127 |
| Creatinine_CURRENT | 0 | 47 | 0.000 |
| GCS_CURRENT | −0.1371 | 10 | −1.371 |
| Lactate_CURRENT | 0.0707 | 6.1 | 0.431 |
| DNA_CHANGE | 0 | −0.5 | 0.000 |
| PROTC_CHANGE | 0 | −35 | 0.000 |
| Platelets_CHANGE | 0 | −245 | 0.000 |
| Creatinine_CHANGE | 0.00219 | −207 | −0.453 |
| GCS_CHANGE | 0 | 0 | 0.000 |
| Lactate_CHANGE | 0.0867 | 3.3 | 0.286 |
| Chronic_Lung_Disease | 0.7839 | 0 | 0.000 |
| Brain_Injury | 1.3733 | 0 | 0.000 |
| Age | 0.0172 | 61 | 1.049 |
| DAYS_IN_ICU_HPT | −0.0608 | 11 | −0.669 |
| LN(DAYS_IN_ICU_HPT) | 1.0503 | 2.3979 | 2.519 |

| | |
|---|---|
| Predicted Ln(Hazard) = B′X | −2.080 |
| Predicted Hazard = Exp(B′X) | 0.125 |
| Predicted Probability of Dying on Day 11 | 0.120 |
| Predicted Probability of Dying in 28 Days | 0.970 |
| Patient ID = 11023 (female) | |

### Example 2—Fitting the CLOGLOG Model to a Refined Input Data Matrix and Estimated Coefficients

A small flaw in the SAS program for imputation was fixed, resulting in the addition of one more septic patient without missing values for the explanatory variables. Consequently, the number of observations of the input data matrix was increased to 6,724 from 356 patients. The number of patients with missing values was: 2 patients for cfDNA, 3 patients for protein C, 1 patient each for platelets and creatinine, and 32 patients for lactate. The proportion of non-survivors remained at 24% after the removal of the patients with missing values. Table 4 shows the estimation results of the day 1 and current specifications of the CLOGLOG model for these 356 patients without missing values. The day 1 specification combines the day 1 variable with the change variable whereas the current specification combines the current variable with the change variable. In both specifications, the level and/or change variables of three TVBIs (cfDNA, lactate, and creatinine) have positive estimated coefficients, indicating that higher values of these variables are associated with greater hazards of dying. In contrast, the estimated coefficients for the corresponding variables of protein C, platelets, and GCS are negative, indicating the opposite association with the hazard of dying. The estimated coefficients of two preconditions (chronic lung disease and previous brain injury) as well as age were also positive, suggesting that the presence of these preconditions as well as advanced age increase the hazard of dying.

TABLE 4

| Explanatory | Estimated | Standard | | Estimated | Standard | |
|---|---|---|---|---|---|---|
| Variable | Coefficient | Error | p-value | Coefficient | Error | p-value |
| | Day 1 Specification | | | Current Specification | | |
| Intercept | −1.6867 | 1.1583 | 0.1453 | −2.0612 | 1.0894 | 0.0585 |
| 1. LEVEL EFFECTS | | | | | | |
| cfDNA_Day_1 | 0.1857 | 0.0382 | <0.0001 | 0.1930 | 0.0370 | <0.0001 |
| Log(ProteinC_Day_1) | −0.7289 | 0.1797 | <0.0001 | −0.6804 | 0.1680 | <0.0001 |
| Log(Platelets_Day_1) | −0.4426 | 0.1655 | 0.0075 | −0.3915 | 0.1159 | 0.0007 |
| Log(Creatinine_Day_1) | — | — | — | — | — | — |
| GCS_Day_1 | −0.1281 | 0.0342 | 0.0002 | −0.1400 | 0.0280 | <0.0001 |
| Lactate_Day_l | 0.0661 | 0.0338 | 0.0504 | 0.0702 | 0.0305 | 0.0212 |
| 2. CHANGE EFFECTS | | | | | | |
| cfDNA_simple_change | 0.1871 | 0.0638 | 0.0034 | — | — | — |
| Log(ProteinC_change) | −0.6341 | 0.2266 | 0.0051 | — | — | — |
| Log(Platelets_change) | −0.3386 | 0.1479 | 0.0221 | — | — | — |
| Log(Creatinine_change) | 0.5919 | 0.1873 | 0.0016 | 0.5274 | 0.1819 | 0.0037 |
| GCS_simple_change | −0.1518 | 0.0286 | <0.0001 | — | — | — |
| Log(Lactate_change) | 0.7902 | 0.1845 | <0.0001 | 0.5013 | 0.1761 | 0.0044 |
| 3. CONTEXT | | | | | | |
| Chronic Lung Disease | 1.0210 | 0.2561 | <0.0001 | 0.9307 | 0.2580 | 0.0003 |
| Previous Brain Injury | 1.1211 | 0.5046 | 0.0263 | 1.1825 | 0.4967 | 0.0173 |
| Age | 0.0152 | 0.0087 | 0.0822 | 0.0151 | 0.0083 | 0.0691 |
| Duration | −0.0666 | 0.0412 | 0.1056 | −0.0625 | 0.0408 | 0.1254 |
| Log(Duration) | 1.1033 | 0.3760 | 0.0033 | 1.0694 | 0.3721 | 0.0041 |

| | | |
|---|---|---|
| AUC | 0.903 (95% CI, 0.864-0.941) | 0.900 (95% CI, 0.861-0.940) |

Although the p-values associated with the variable “Duration” in Table 4 were somewhat large (0.106 in the day 1 specification and 0.125 in the current specification), it was chosen not to set its coefficient to 0 for the following reason. Since “Log(Duration)” is a monotonically increasing function of “Duration”, they must be positively correlated. This correlation contributed to the inflation of the standard error of the estimated coefficient of “Duration” and hence the inflation of the p-value. The dotted dark and light grey curves in FIG. 2 were generated by the day 1 and current specifications of the CLOGLOG model, respectively. The curves show that there is an unusually low hazard of dying on days 1 and 2 followed by an increase in the hazard of dying during days 3 to 5. This pattern likely reflects the effect of an eligibility criterion for inclusion into the study (ie. patients are to be excluded from the study if they are not expected to remain in the ICU for ≥72 hours). In general, the temporal pattern of the hazard is hard to interpret, because it absorbs the effects of the balance of various selection biases, such as (1) the exclusion of patients who were not expected to remain in the ICU for ≥72 hours, (2) the temporal decline in the number of sicker patients due to the death process, and (3) the temporal decline in the number of healthier patients due to the discharge process. The decline of the hazard after day 7 likely reflects the beneficial effects of the treatments and care received by the patients in ICU.

Keeping selection biases in mind, it is not surprising that the sharp rise in the hazard from day 1 to day 5 is inconsistent with the fact that the daily averages of five of the six TVBIs improved during the same time interval: cfDNA decreased by 3.8%, protein C increased by 25.5%, creatinine decreased by 19.4%, GCS increased by 18.0%, and lactate decreased by 30.7%. This inconsistency was resolved in the CLOGLOG model by the positive estimated coefficient of “log(duration)”. It is noted that the omission of both duration and log(duration) from the Day 1 specification of the model led to the effects of most TVBIs being underestimated or, in the case of the day 1 variable of lactate, even non-significant (p=0.221). This finding reveals a shortcoming of the partial-likelihood approach commonly adopted by users of the Cox model: as a consequence of removing any function of time, the estimated effects of some TVBIs became misleading. In other words, the partial-likelihood approach is associated with a high risk of making misleading inferences from observational data which are subject to various selection biases. This finding also reveals that the above-mentioned eligibility criterion should not be used any more. It is worth noting that among the patients contributing to the input data, one died on day 1, 4 died on day 2, and 8 died on day 3, suggesting that some clinicians realized that this criterion had the undesirable effect of resulting in not only selection bias but also loss of valuable information and hence chose not to use it. FIG. 2 also shows circles representing the so-called “observed daily hazards of dying”, although hazards are not directly observable. They were computed from the longitudinal data according to the method used for constructing the Kaplan-Meier survival curves, without using any model. The irregular scatter of the circles suggests that predicting the mortality outcome of a patient on any given day is a difficult task.

Since the dependent variable in this CLOGLOG model is the daily hazard of dying, the logistic procedure of SAS automatically uses the predicted daily probabilities of dying for all records (person-days) to conduct the ROC analysis and compute the value of AUC. In other words, the value of AUC was computed by using the daily probability of dying as the classifier. For the day 1 and current specifications of the CLOGLOG model, it yielded the values of 0.865 (95% CI, 0.826-0.903) and 0.866 (95% CI, 0.828-0.904) for AUC. These face values should not be compared with the AUC values computed in other studies that used a logistic model with the dependent variable being the probability of dying in 28 days because in those studies the probability of dying in 28 days was used as the classifier. Simulations that compared the AUC values computed by the two classifiers (the daily probability of dying versus the probability of dying in 28 days) were conducted for the same set of the daily hazards of dying. It was found that the former was markedly smaller than the latter in most cases. An explanation for this large difference is that predicting whether a patient would die on a given day is more difficult than predicting whether a patient would die in 28 days. The simulations also indicate that the difference in the face values of AUC between the two classifiers tends to become larger when the level of hazards is raised and when the predictive power of the model becomes stronger.

To make the face values of AUC comparable to those in other studies, the AUC was recomputed in the following way. From the large input file of person-day records, the record of the last day for each patient was selected. For each patient, the selected record was used to compute his/her predicted daily hazard of dying, based the estimated coefficients of the CLOGLOG model. The predicted hazards were then transformed into the predicted probabilities of dying in 28 days by the formula 1−e^{-28H}^{ᵢ} where Hᵢ was the ith patient's predicted daily hazard of dying. These predicted probabilities were then used to conduct the ROC analysis and compute the value of the AUC, with the two specifications having almost identical predictive powers. The AUC based on the use of the probability of dying within 28 days of ICU admission as the classifier was 0.903 (95% CI, 0.864-0.941) for the day 1 specification and 0.900 (95% CI, 0.861-0.940) for the current specification. However, the two specifications revealed different biological insights. The day 1 specification revealed that for most TVBIs the change variable had a stronger predictive power than the day 1 variable. The current specification revealed that for each TVBI, the predictive powers of the day 1 and change variables were mostly inherited by the current variable, and that the most up-to-date information on creatinine and lactate should be complemented by the information on their changes for achieving a high predictive power.

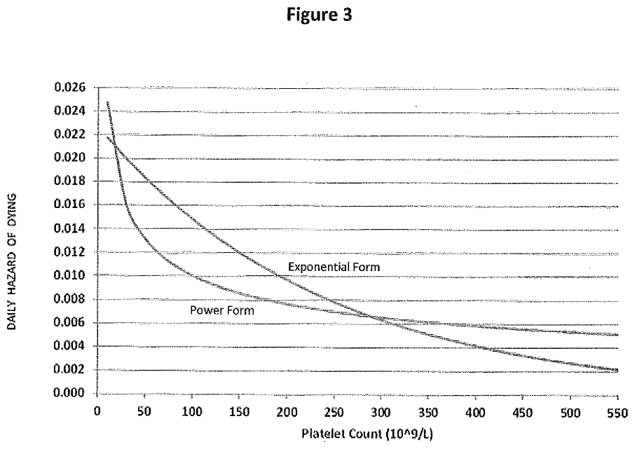

To enhance the model's predictive power and to obtain biologically meaningful insights in regard to changes in TVBI values, the day 1 as well as the current variables of protein C, platelet count, and creatinine were log-transformed as their effects on mortality differences among patients tend to become negligible for patients with scores higher than the normal level. Mathematically, this transformation implies the replacement of an exponential function by a power function in the dependence of the hazard on the level variable in question. Since power function has a flatter tail than does exponential function, the negligible effects are better represented but the former. FIG. 3 shows the difference between these two functions for the current variable of platelet count in the current specification of the CLOGLOG model. In drawing the curves, the values of all other variables in the model were set at the mean of all patients. The shapes of the two functions differ markedly. The power function has a greater curvature and a flatter tail, suggesting that variations in platelet counts above 250 have little effect on the risk of dying.

The model's predictive power was further enhanced by replacing the simple difference between current and day 1 variables of some TVBIs with proportional changes. Let X₁ be the day 1 variable, and Xₜ be the current variable of the TVBI in question. This alternative specification of its change variable is

$$ln ( \frac{{X}_{t}}{{X}_{1}} ) = ln ( 1 + Δ X / {X}_{1} ) , where \frac{Δ X}{{X}_{1}}$$

is the proportional change. Mathematically, the switch to this alternative implies the replacement of the exponential function e^{βΔX} by the power function (1+ΔX/X₁)^{β}, where β is an unknown coefficient to be estimated. In this model, the change factor is more suitable than the simple change for representing the change variables of protein C, platelets, creatinine, and lactate. In other words, proportional change is better than simple change for quantifying the change effects of these 4 TVBIs.

An important methodological issue is whether the level variable of any TVBI had a non-monotonic effect. For example, in the construction of various versions of APACHE, several variables such as body temperature were assumed to have non-monotonic effects. To deal with this issue, by expanding from the best day 1 specification of the CLOGLOG model reported in Table 4, the level effect of each TVBI was quantified by two variables simultaneously: its day 1 variable and the log of the day 1 variable. The combination of these two variables provided a flexibility to allow the data to decide whether the TVBI in question had a non-monotonic effect, which was to be revealed by the two variables having coefficients with opposite signs. This was done for each TVBI in turn. The signs and p-values associated with the two variables were: same sign for cfDNA, with p=0.28 and 0.34; same sign for protein C, with p=0.47 and 0.21; opposite signs for platelets, with p=0.71 and 0.10; same sign for GCS, with p=0.41 and 0.85; opposite signs for lactate, with p=0.21 and 0.90. Thus, it can be inferred that within the observed data range, none of the TVBIs had a significant non-monotonic effect.

The finding that the estimated coefficients of the day 1 and current variables of creatinine are not significantly different from 0 warrants explanation. This finding was mainly due to the overlap of their weaker predictive powers with the stronger predictive powers of the day 1 and current variables of protein C and platelets. Panel 1 of Table 5 shows that in the context of the preconditions of chronic lung disease and previous brain injury, age, and duration, the day 1 variable of creatinine, with p=0.1002, did not have a significant effect on the mortality hazard, although its estimated coefficient had the biologically meaningful positive sign. This finding is misleading because the day 1 and change variables of creatinine had a strong negative correlation (r=−0.57, N=6,724, p<0.001). This negative correlation implied that large improvements in creatinine occurred mostly to patients whose initial values were relatively poor (high), so that the pool of survivors contained increasingly higher proportion of patients whose creatinine was relatively poor on day 1 but had experienced large improvement (reduction) afterwards. To control for the distorting effect of this selective improvement, the proper assessment of the effect of the day 1 variable required the simultaneous inclusion of the corresponding change variable into the model. Since strong negative correlation between day 1 and change variables occurred to all six TVBIs, the use of the day 1 variable of each TVBI should be accompanied by the corresponding change variable. Otherwise, the effect of the day 1 variable will be understated. Panel 2 of Table 5 shows that the addition of the change variable of creatinine not only raised the AUC markedly from 0.623 to 0.693 but also helped make the corresponding day 1 variable highly significant (p=0.0036) and caused its estimated coefficient to increase markedly from 0.2345 to 0.4320. Thus, controlling for the selective improvement, patients with relatively high day 1 creatinine were found to be at higher risk of dying. However, panel 3 of Table 5 shows that the further addition of the day 1 and change variables of protein C and platelets caused the day 1 variable of creatinine to become a non-significant explanatory variable (coefficient=0.1593, p=0.3231). This was the consequence of the overlap between the weaker predictive power of the day 1 variable of creatinine and the stronger predictive powers of the day 1 variables of protein C and platelets. Behind this overlap were the significant correlations of the day 1 variable of creatinine with the corresponding variables of protein C and platelets. The two correlations were −0.155 (N=6,724, p<0.0001) for protein C and −0.202 (N=6,724, p<0.0001) for platelets. In short, the loss of usefulness of the day 1 variable of creatinine resulted from a multicollinearity problem.

However, it is useful to remember from Panel 2 that a strong correlation between explanatory variables need not result in a multicollinearity problem.

TABLE 5

| | | Wald | | | Wald | | | Wald | |
|---|---|---|---|---|---|---|---|---|---|
| Explanatory | Estimated | Chi- | | Estimated | Chi- | | Estimated | Chi- | |
| Variable | Coefficient | Square | p-value | Coefficient | Square | p-value | Coefficient | Square | p-value |
| | Panel 1 | | | Panel 2 | | | Panel 3 | | |
| Intercept | −6.6784 | 51.9 | <.0001 | 0.0035 | 0.0 | 0.9979 | −0.0730 | 0.0 | 0.951 |
| 1. LEVEL EFFECTS | | | | | | | | | |
| cfDNA Day_1 | — | — | — | — | — | — | — | — | — |
| Log(ProteinC_Day_1) | — | — | — | — | — | — | −0.8743 | 24.9 | <.000 |
| Log(Platelets_Day_1) | — | — | — | — | — | — | −0.6345 | 16.0 | <.000 |
| Log(Creatinine_Day_1) | 0.2345 | 2.7 | 0.1002 | 0.4320 | 8.5 | 0.0036 | 0.1593 | 1.0 | 0.323 |
| GCS_Day_1 | — | — | — | — | — | — | — | — | — |
| Lactate_Day_1 | — | — | — | — | — | — | — | — | — |
| 2. CHANGE EFFECTS | | | | | | | | | |
| cfDNA_simple_change | — | — | — | — | — | — | — | — | — |
| Log(ProteinC_change) | — | — | — | — | — | — | −1.0538 | 23.0 | <.000 |
| Log(Platelets_change) | — | — | — | — | — | — | −0.4700 | 9.9 | 0.001 |
| Log(Creatinine_change) | — | — | — | 0.9314 | 21.9 | <.0001 | 0.6196 | 8.6 | 0.003 |
| GCS_simple_change | — | — | — | — | — | — | — | — | — |
| Log(Lactate_change) | — | — | — | — | — | — | — | — | — |
| 3. CONTEXT | | | | | | | | | |
| Chronic Lung Disease | 0.5566 | 5.7 | 0.0169 | 0.5843 | 6.2 | 0.0125 | 1.0731 | 18.8 | <.000 |
| Previous Brain Injury | 0.9738 | 4.4 | 0.0369 | 1.0451 | 5.0 | 0.0251 | 1.5823 | 10.7 | 0.001 |
| Age | 0.0126 | 2.8 | 0.0953 | 0.0085 | 1.2 | 0.2673 | 0.0101 | 1.6 | 0.209 |
| Duration | −0.0754 | 3.7 | 0.0538 | −0.0821 | 4.4 | 0.0366 | −0.0660 | 2.8 | 0.096 |
| Log(Duration) | 0.4762 | 2.1 | 0.1500 | 0.5629 | 2.9 | 0.0886 | 0.6332 | 3.4 | 0.063 |

| | | | |
|---|---|---|---|
| AUC | 0.623 (95% CI, 0.563-0.682) | 0.693 (95% CI, 0.634-0.752) | 0.800 (95% CI, 0.750-0.850 |

In this table, the computation of AUC is based on the use of the daily probability of dying as the classifier.

An important feature of the current variable of a TVBI is that it could inherit the predictive powers of the corresponding day 1 and change variables. In Panel 1 of Table 6 where the current variable of creatinine was included in the CLOGLOG model with the same set of contextual variables, it had a highly significant effect (coefficient=0.5673, Chi-square=17.2, p<0.0001), as expected. In Panel 2 of Table 6 where the change variable of creatinine was added to the model, the effect of the current variable of creatinine was weakened (coefficient-0.4320, Chi-square=8.5, p=0.0036), because part of its inherited predictive power was taken back by the change variable. The correlation between the current and change variables of creatinine was 0.351 (p<0.0001). In Panel 3 of Table 6 where the current variables of protein C and platelets were further added to the model, the effect of the current variable of creatinine was markedly reduced and became non-significant (coefficient-0.1560, Chi-square-1.0, p=0.3245), because its weaker predictive power overlapped to a large extent with the stronger predictive powers of the current variables of protein C and platelets. The correlation of the former variable with the two latter variables were −0.163 (p<0.0001) and −0.300 (p<0.0001), respectively. In short, the current variable of creatinine lost its usefulness after encountering the multicollinearity problem twice.

TABLE 6

| | | Wald | | | Wald | | | Wald | |
|---|---|---|---|---|---|---|---|---|---|
| Explanatory | Estimated | Chi- | | Estimated | Chi- | | Estimated | Chi- | |
| Variable | Coefficient | Square | p-value | Coefficient | Square | p-value | Coefficient | Square | p-value |
| | Panel 1 | | | Panel 2 | | | Panel 3 | | |
| Intercept | −8,1561 | 81.3 | <.0001 | −7.3796 | 60.4 | <.0001 | −0.0730 | 0.0 | 0.9518 |
| 1. LEVEL EFFECTS | | | | | | | | | |
| cfDNA_current | — | — | — | — | — | — | — | — | — |
| Log(ProteinC_current) | — | — | — | — | — | — | −0.9396 | 35.6 | <.0001 |
| Log(Platelets_eurrent) | — | — | — | — | — | — | −0.5516 | 23.4 | <.0001 |
| Log(Creatinine_current) | 0.5673 | 17.2 | <.0001 | 0.4320 | 8.5 | 0.0036 | 0.1560 | 1.0 | 0.3245 |
| GCS_current | — | — | — | — | — | — | — | — | — |
| Lactate_current | — | — | — | — | — | — | — | — | — |
| 2. CHANGE EFFECTS | | | | | | | | | |
| cfDNA_simple_change | — | — | — | — | — | — | — | — | — |
| Log(ProteinC_change) | — | — | — | — | — | — | — | — | — |
| Log(Platelets_change) | — | — | — | — | — | — | — | — | — |
| Log(Creatinine_change) | — | — | — | 0.4994 | 5.8 | 0.0164 | 0.4423 | 4.5 | 0.0341 |
| GCS_simple_change | — | — | — | — | — | — | — | — | — |
| Log(Lactate_change) | — | — | — | — | — | — | — | — | — |
| 3. CONTEXT | | | | | | | | | |
| Chronic Lung Disease | 0.6081 | 6.8 | 0.0093 | 0.5843 | 6.2 | 0.0125 | 1.0637 | 18.6 | −1.0001 |
| Previous Brain Injury | 1.0755 | 5.3 | 0.0209 | 1.0451 | 5.0 | 0.0251 | 1.5955 | 11.0 | 0.0009 |
| Age | 0.0097 | 1.6 | 0.2072 | 0.0085 | 1.2 | 0.2673 | 0.0090 | 1.4 | 0.2365 |
| Duration | −0.0808 | 4.2 | 0.0396 | −0.0821 | 4.4 | 0.0366 | −0.0628 | 2.6 | 0.1102 |
| Log(Duration) | 0.5564 | 2.8 | 0.0930 | 0.5629 | 2.9 | 0.0886 | 0.5985 | 3.1 | 0.0760 |

| | | | |
|---|---|---|---|
| AUC | 0.676 (95% CI, 0.619-0.734) | 0.693 (95% CI, 0.634-0.752) | 0.799 (95% CI, 0.749-0.848) |

In this table, the computation of AUC is based on the use of the daily probability of dying as the classifier.

### Example 3—Relative Importance Among Wills and Each Level and Change Variable

The hazard ratios computed from the estimated coefficients by exponentiation are not suitable for comparing the relative importance of the explanatory variables because the explanatory variables do not share a common unit. To overcome the comparability problem, a new way for assessing the relative importance of the explanatory variables was introduced by transforming the CLOGLOG model into the following form:

log(Hᵢₜ)=β₀+β₁Xᵢ₁ₜ+β₂Xᵢ₂ₜ+ . . . +βₖXᵢₖₜ+ . . . (7)

where βₖ is the kth element of β′ and X_{iKt} is the kth element of Xᵢₜ. Despite the fact that the explanatory variables have different physical units, the additive terms on the right-hand-side of Eq. (7) have a common unit, log(1/day), so that their magnitudes can be used to evaluate the relative importance among the explanatory variables in determining the log of hazard. Thus, βₖXᵢₖₜ is called the additive contribution to the log of hazard of dying by the kth explanatory variable.

The additive contributions to two representative log of hazards was then computed: (1) the predicted log of hazard computed for the mean of the non-survivor group, and (2) the predicted log of hazard computed for the mean of the survivor group. For each explanatory variable, the difference in its additive contributions to these two representative log of hazards is then considered as its predictive power in distinguishing non-survivors from survivors: the greater the difference, the greater the predictive power.

For the day 1 specification of the model, Table 7 demonstrates the computation of the difference in the additive contributions to the log of hazard between (1) the mean of non-survivors and (2) the mean of survivors for each explanatory variable. For example, the means of cfDNA on day 1 were 6.126 μg/mL for non-survivors and 4.705 μg/mL for survivors. By multiplying these two means by the estimated coefficient of 0.1857, it was found that the additive contributions of the day 1 variable of cfDNA to the log of hazard were 1.138 for non-survivors and 0.874 for survivors. Hence, the predictive power of the day 1 variable of cfDNA was 1.138−0.874=0.264, which is shown in the last column of the table. Such computations were done for all explanatory variables in the table. From the last column, with respect to day 1 variables, cfDNA had the greatest predictive power (0.246). With respect to change variables, GCS had the greatest predictive power (0.701).

TABLE 7

| | | | Additive | | Additive | | |
|---|---|---|---|---|---|---|---|
| | | Values of | Contribution | Values of | Contribution | Values of | |
| Explanatory | Estimated | Explanatory | to Log of | Explanatory | to Log of | Explanatory | Predictive |
| Variable | Coefficient | Variable | Hazard | Variable | Hazard | Variable | Power |
| | | For Mean of Non-Survivors | | For Mean of Survivors | | Difference | |
| | Vector ″B″ | Vector ″X″ | Terms in B′X | Vector ″X″ | Terms in B′X | Vector ″X″ | Terms in B′X |
| Intercept | −1.6867 | 1 | −1.687 | 1 | −1.687 | | |
| cfDNA_day_1 | 0.1857 | 6.126 | 1.138 | 4.705 | 0.874 | 1.421 | 0.264 |
| Log(ProteinC_day_1) | −0.7289 | 3.946 | −2.876 | 4.198 | −3.060 | −0.252 | 0.184 |
| Log(Platelets_day_1) | −0.4426 | 5.198 | −2.301 | 5.423 | −2.400 | −0.225 | 0.100 |
| Log(Creatinine_day_1) | 0 | 5.113 | 0.000 | 5.038 | 0.000 | 0.075 | 0.000 |
| GCS_day_1 | −0.1281 | 9.600 | −1.230 | 9.764 | −1.251 | −0.164 | 0.021 |
| Lactate_day _l | 0.0661 | 3.991 | 0.264 | 2.866 | 0.189 | 1.125 | 0.074 |
| cfDNA_simple_change | 0.1871 | 0.025 | 0.005 | −0.122 | −0.023 | 0.148 | 0.028 |
| Log(ProteinC_change) | −0.6341 | 0.079 | −0.050 | 0.316 | −0.200 | −0.237 | 0.150 |
| Log(Platelets_change) | −0.3386 | −0.254 | 0.086 | 0.096 | −0.033 | −0.350 | 0.119 |
| Log(Creatinine_change) | 0.5919 | 0.003 | 0.002 | −0.295 | −0.175 | 0.298 | 0.176 |
| GCS_simple_change | −0.1518 | −0.788 | 0.120 | 3.827 | −0.581 | −4.615 | 0.701 |
| Log(Lactate_change) | 0.7902 | 0.091 | 0.072 | −0.627 | −0.495 | 0.719 | 0.568 |
| Chronic Lung Disease | 1.021 | 0.341 | 0.348 | 0.210 | 0.215 | 0.131 | 0.134 |
| Brain_Injury | 1.1211 | 0.059 | 0.066 | 0.026 | 0.029 | 0.033 | 0.037 |
| Age | 0.0152 | 66.802 | 1.015 | 62.618 | 0.952 | 4.185 | 0.064 |
| Duration | −0.0666 | 10.565 | −0.704 | 21.498 | −1.432 | −10.933 | 0.728 |
| Log(Duration) | 1.1033 | 2.358 | 2.601 | 3.068 | 3.385 | −0.710 | −0.784 |

| | | | |
|---|---|---|---|
| Predicted Ln(Hazard) = B′X | −3.131 | −5.693 | 2.562 |
| Predicted Hazard = EXP(B′X) | 0.044 | 0.003 | 0.040 |
| Predicted Daily Probability of Dying | 0.043 | 0.003 | 0.039 |
| Predicted Probability of Dying in 28 Days | 0.706 | 0.090 | 0.616 |
| Predicted Overall Hazard Ratio | | | 13.0 |

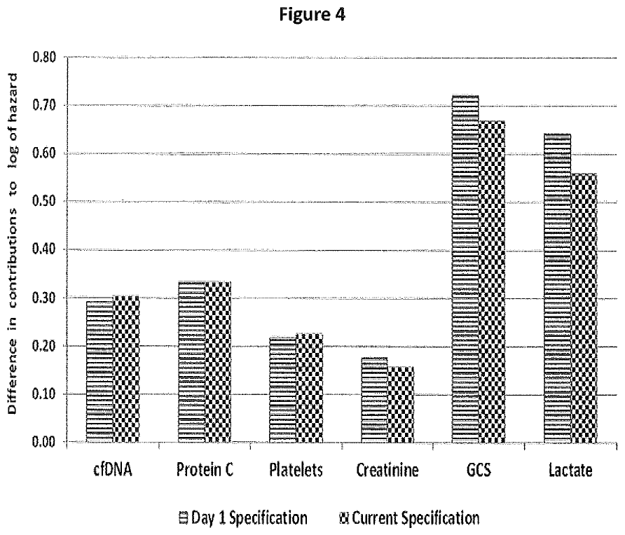

The combined predictive power of the day 1 and change variables of each TVBI was computed by summing the predictive powers of its day 1 and change variables. For example, in the day 1 specification of the model, the combined predictive power of cfDNA was 0.264+0.028=0.292, whereas the combined predictive power of GCS was 0.021+0.701=0.722. In other words, GCS was much more powerful than cfDNA in distinguishing non-survivors from survivors. The predictive power of GCS came mostly from its change, whereas the predictive power of cfDNA came mostly from its initial level. For the current specification of the model, Table 8 shows similar computations for evaluating the relative importance of the TVBIs in terms of their current and change variables. Although most change variables in the current specification had zero contribution to the predictive power, the change variable of lactate, with a predictive power of 0.360, remained important. Since this predictive power was greater than that of its current variable (0.200), recent history was more important than current status for lactate. The combined predictive power of each TVBI in the current specification of the model was also computed. The patterns of the predictive powers of the six TVBIs in two specifications of CLOGLOG model turned out to be similar (FIG. 4).

TABLE 8

| | | | Additive | | Additive | | |
|---|---|---|---|---|---|---|---|
| | | Values of | Contribution | Values of | Contribution | Values of | |
| Explanatory | Estimated | Explanatory | to Log of | Explanatory | to Log of | Explanatory | Predictive |
| Variable | Coefficient | Variable | Hazard | Variable | Hazard | Variable | Power |
| | | For Mean of Non-Survivors | | For Mean of Survivors | | Difference | |
| | Vector ″B″ | Vector ″X″ | Terms in B′X | Vector ″X″ | Terms in B′X | Vector ″X″ | Terms in B′X |
| Intercept | −2.0612 | 1 | −2.061 | 1 | −2.061 | | |
| cfDNA_current | 0.193 | 6.151 | 1.187 | 4.583 | 0.884 | 1.569 | 0.303 |
| Log(ProteinC_current) | −0.6804 | 4.025 | −2.739 | 4.514 | −3.072 | −0.489 | 0.333 |
| Log(Platelets_current) | −0.3915 | 4.944 | −1.936 | 5.519 | −2.161 | −0.575 | 0.225 |
| Log(Creatinine_current) | 0 | 5.116 | 0.000 | 4.744 | 0.000 | 0.372 | 0.000 |
| GCS_current | −0.14 | 8.812 | −1.234 | 13.590 | −1.903 | −4.779 | 0.669 |
| Lactate_current | 0.0702 | 4.373 | 0.307 | 1.531 | 0.107 | 2.842 | 0.200 |
| cfDNA_simple_change | 0 | 0.025 | 0.000 | −0.122 | 0.000 | 0.148 | 0.000 |
| Log(ProteinC_change) | 0 | 0.079 | 0.000 | 0.316 | 0.000 | −0.237 | 0.000 |
| Log(Platelets_change) | 0 | −0.254 | 0.000 | 0.096 | 0.000 | −0.350 | 0.000 |
| Log(Creatinine_change) | 0.5274 | 0.003 | 0.002 | −0.295 | −0.155 | 0.298 | 0.157 |
| GCS_simple_change | 0 | −0.788 | 0.000 | 3.827 | 0.000 | −4.615 | 0.000 |
| Log(Lactate_change) | 0.5013 | 0.091 | 0.046 | −0.627 | −0.314 | 0.719 | 0.360 |
| Chronic Lung Disease | 0.9307 | 0.341 | 0.318 | 0.210 | 0.196 | 0.131 | 0.122 |
| Brain_Injury | 1.1825 | 0.059 | 0.070 | 0.026 | 0.031 | 0.033 | 0.039 |
| Age | 0.0151 | 66.802 | 1.009 | 62.618 | 0.946 | 4.185 | 0.063 |
| Duration | −0.0625 | 10.565 | −0.660 | 21.498 | −1.344 | −10.933 | 0.683 |
| Log(Duration) | 1.0694 | 2.358 | 2.521 | 3.068 | 3.281 | −0.710 | −0.760 |

| | | | |
|---|---|---|---|
| Predicted Ln(Hazard) = B′X | −3.171 | −5.565 | 2.394 |
| Predicted Hazard = EXP(B′X) | 0.042 | 0.004 | 0.038 |
| Predicted Daily Probability of Dying | 0.041 | 0.004 | 0.037 |
| Predicted Probability of Dying in 28 Days | 0.691 | 0.102 | 0.589 |
| Predicted Overall Hazard Ratio | | | 11.0 |

As shown in FIG. 4, GCS is about four times as important as creatinine. This finding raises a question about the appropriateness of giving equal weight to GCS and creatinine in the creation of MODS and SOFA. Since lactate, cfDNA, and protein C were not part of MODS and SOFA but were found to have rather strong predictive powers, it is likely that the combination of the chosen TVBIs would outperform both MODS and SOFA. From FIG. 4, it was also found that the six TVBIs could be divided into 3 groups in terms of the predictive powers: (1) GCS (0.72 in day 1 specification and 0.67 in current specification) and lactate (0.64 and 0.56) on the top; (2) protein C (0.33 and 0.33) and cfDNA (0.29 and 0.30) in the middle; and (3) platelets (0.22 and 0.23) and creatinine (0.18 and 0.16) at the bottom.

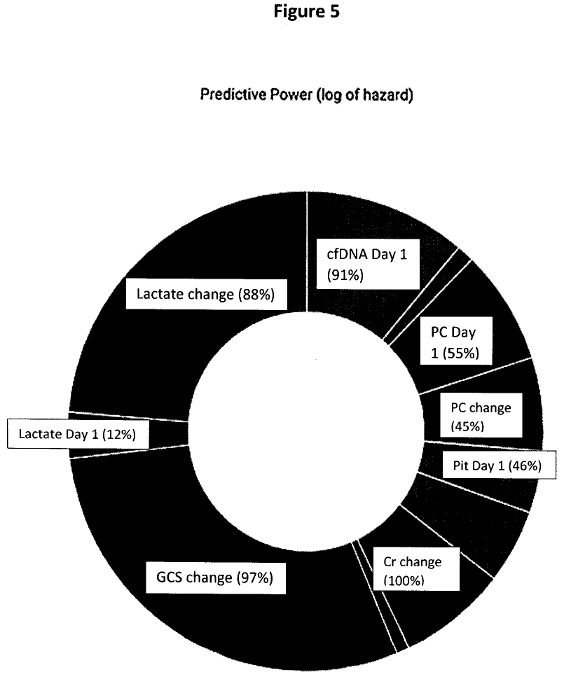

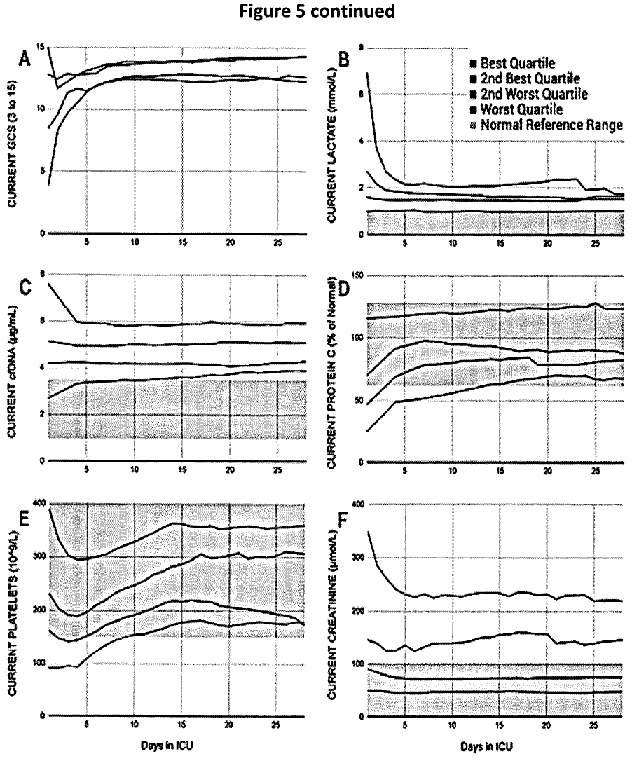

The information in the last columns of Tables 7 and 8 was used to assess the relative importance of the day 1 or current variable against the corresponding change variable for each TVBI and create the top panel of FIG. 5. As shown in the top panel of FIG. 5, the relative predictive powers between the day 1 and change variables differed markedly among the six TVBIs in the day 1 specification of the model. For example, 88% of the predictive power of lactate came from its change variable whereas 91% of the predictive power of cfDNA came from its day 1 variable. To gain more insights into this contrast, the temporal patterns of all six TVBIs were examined (FIG. 5, graphs A to F).

One temporal attribute shared by all six TVBI was that the day 1 variable had a strong negative correlation with the corresponding change variable (r=−0.80 for lactate, −0.70 for GCS, −0.58 for cfDNA, −0.57 for creatinine, −0.36 for platelets, and −0.27 for protein C; N=6,724). This attribute suggested that sicker patients on day 1 tended to benefit more from treatments and to experience greater improvement. Keeping this general attribute in mind, for gaining insights into the temporal pattern of each TVBI, the septic patients were divided into four quartile groups in terms of the day 1 variable, and then examined the trend of the daily averages of each group. To avoid the selection bias resulting from the death process that could misleadingly exaggerate improvements as the sickest patients in each group were successively removed, the daily records of all non-survivors were removed from the data before the daily averages were calculated. Except for the less clear evidence for platelets, the worst quartile group experienced the greatest improvement for each TVBI. To see the effects of removing non-survivors, the temporal graphs in FIG. 1 and the corresponding temporal graphs generated without the removal of non-survivors from the sample were compared and, as expected, the failure to remove non-survivors resulted in the exaggeration of improvements.

From the differences in predictive powers and temporal patterns, it is inferred that current management strategies produce a rapid improvement in some TVBIs (e.g. lactate, GCS) which contribute to a reduction in ICU mortality. However, the levels of some TVBIs do not change significantly over time (e.g. cfDNA, protein C), suggesting that therapeutic strategies to reduce cfDNA levels or restore protein C levels warrant further investigations. Being the two TVBIs with the greatest predictive powers, GCS and lactate turned out to show the greatest improvements: the average of the worse quartile group experienced a sharp and rapid improvement, and the averages of all quartile groups converged to a narrow range around a low risk level. This temporal pattern is consistent with the finding that most of the predictive powers of GCS and lactate (97% and 88%) come from their change variables. This finding suggests that the treatments that helped improve GCS and lactate contributed greatly to the reduction of the mortality risk level. For cfDNA, the average of the worst quartile group remained at the high risk level of 6 ug/mL, after a brief improvement from day 1 to day 4, while the average of the best quartile group increased from less than 3 ug/mL to nearly 4 ug/mL. This temporal pattern corresponded to the finding that a very high proportion of the predictive power (91%) of cfDNA came from its day 1 variable, leaving only 9% for its change variable. This finding suggests that novel strategies for reducing cfDNA can make an important contribution to improving mortality outcome.

Protein C was the third most powerful predictor of the hazard of dying, with 45% of its predictive power coming from its change variable. There was a general pattern of improvement in protein C for all quartile groups, with the worst quartile group experiencing the greatest improvement. However, by day 28, the gap between the worst and best quartile groups remained quite large, with the average of the worst quartile group being <70% of the normal level. Compared with lactate and GCS, the improvement in protein C was smaller and more prolonged, so that its contribution to the overall reduction of mortality risk was much less. This temporal pattern corresponded to the finding that less than half of the predictive power of protein C came from its change variable. This finding suggests that increasing protein C can also make an important contribution to improving mortality outcome.

Platelet counts had the second weakest predictive power, with 54% of it coming from the change variable. It has the distinctive feature of a general lack of improvement during the first few days. During the first 3 or 4 days, the average of the worst quartile group remained at the same high risk level of about 100 units, whereas the averages of the 3 better quartile groups all worsened. Beyond day 4, the averages of all quartile groups experienced a prolonged and moderate improvement until around the end of the second week. By day 28, the gap between the worst quartile group (175) and the best quartile group (350) remained large.

Creatinine, the weakest predictor, had all of its predictive power coming from its change variable. The average creatinine of the worst quartile group experienced an improvement from 350 μmol/L on day 1 to 225 μmol/L on day 6. However, with almost no further improvement, the gap between the worst quartile group (220 μmol/L) and the best quartile group (50 μmol/L) remained large until day 28. In summary, the limited improvement in creatinine made a small contribution to the overall reduction in mortality risk.

In the current specification, it was found that the current variable accounted for 100% of the predictive powers of cfDNA, protein C, platelets, and GCS, and that the change variable accounted for 64% of lactate's predictive power, which was less than the 88% in the day 1 specification of the model. These findings implied that for most TVBIs, the predictive powers of the day 1 and change variables are inherited by the corresponding current variables. Note that the reason for the change variable to represent 100% of creatinine's predictive power in both specifications was that the weaker predictive powers of the day 1 and change variables of creatinine overlapped with the stronger predictive powers of the day 1 and change variables of protein C and platelets.

### Example 4—Differences Between Septic and Non-Septic Patients

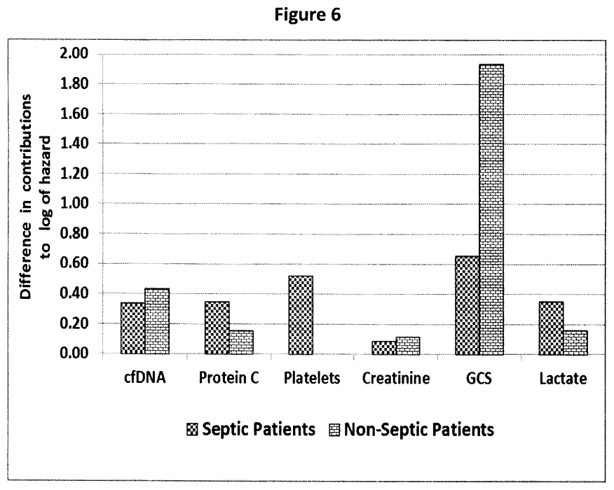

Using the input data from the original 355 septic patients, the relative importance of the 6 indicators is shown in Table 9. In terms of the difference in the contributions to the log of hazard between the non-survivors and survivors by the current indicators, GCS (0.655), platelets (0.518), cfDNA (0.335), and protein C (0.343) were more important than lactate (0.201) and creatinine (0) for septic patients. In contrast, GCS (1.932) was the most important indicator for non-septic patients, followed by cfDNA (0.433), lactate (0.159) and creatinine (0.112). An advantage of this additive index over the hazard ratio is that it can reflect the combined effect of the current and change variables for each TVBI. Such combined effects are shown in FIG. 6, where GCS turned out to be most important for both septic and non-septic patients. The main difference between septic and non-septic patients was the relative importance of platelets and protein C for the former, and the overwhelming importance of GCS for the latter.

TABLE 9

Probability

| | of Dying | | | | | | | | | | |
|---|---|---|---|---|---|---|---|---|---|---|---|
| | | In 28 | (Current) | | | | | | (Change) | | |
| Predicted Hazard | Daily | Days | cfDNA | Protein C | Platelets | Creatinine | GCS | Lactate | Lactate | Creatinine | Protein |

PANEL A: SEPTIC PATIENTS
Additive terms of the natural log of the predicted hazard (common unit: ln(1/day))

| Group 1: Patients who died (N = 85; 24%) | | | | | | | | | | | | |
|---|---|---|---|---|---|---|---|---|---|---|---|---|
| MEAN | 0.053 | 0.051 | 0.77 | 1.291 | −0.548 | −0.662 | — | −1.208 | 0.309 | 0.033 | 0.001 | — |
| Group 2: Patients who survived (N = 270; 76%) | | | | | | | | | | | | |
| MEAN | 0.004 | 0.004 | 0.11 | 0.956 | −0.891 | −1.180 | — | −1.863 | 0.108 | −0.116 | −0.085 | — |
| Group 1-Group 2: | | | | | | | | | | | | |
| MEAN | | | | 0.335 | 0.343 | 0.518 | — | 0.655 | 0.201 | 0.149 | 0.086 | — |
| RANK | | | | 4 | 3 | 2 | | 1 | 5 | 6 | 7 | |

Values of Explanatory Variables

| | | | | | | | | |
|---|---|---|---|---|---|---|---|---|
| Group 1: Patients who died | | | | | | | | |
| MEAN | 6.2 | 56.0 | 140.4 | — | 8.8 | 4.4 | 0.4 | 0.5 |
| Group 2: Patients who survived | | | | | | | | |
| MEAN | 4.6 | 91.1 | 250.2 | — | 13.6 | 1.5 | −1.3 | −39.0 |
| Group 1-Group 2: | | | | | | | | |
| Percentage Difference** | 29.8 | −47.7 | −56.2 | — | −42.6 | | | |

PANEL B: NON-SEPTIC PATIENTS
Additive terms of the natural log of the predicted hazard (common unit: 1n(1/day))

| Group 1: Patients who died (N = 52, 18.1%) | | | | | | | | | | | | |
|---|---|---|---|---|---|---|---|---|---|---|---|---|
| MEAN | 0,064 | 0.062 | 0.83 | 1.290 | — | — | 0.327 | −2.078 | 0.346 | — | — | 0.013 |
| Group 2: Patients who survived (N = 236; 81.9%) | | | | | | | | | | | | |
| MEAN | 0.007 | 0.006 | 0.17 | 0.857 | — | — | 0.215 | −4.010 | 0.187 | — | — | −0.14 |
| Group 1-Group 2: | | | | | | | | | | | | |
| MEAN | | | | 0.433 | — | — | 0.112 | 1.932 | 0.159 | — | — | 0.154 |
| RANK | | | | 2 | | | 5 | 1 | 3 | | | 4 |

Values of Explanatory Variables

| | | | | | | | |
|---|---|---|---|---|---|---|---|
| Group 1: Patients who died | | | | | | | |
| MEAN | 6.3 | 155.1 | 7.0 | 3.0 | — | — | −1.4 |
| Group 2: Patients who survived | | | | | | | |
| MEAN | 4.2 | 102.1 | 13.4 | 1.6 | — | — | 14.5 |
| Group 1-Group 2: | | | | | | | |
| Percentage Difference** | 40.3 | 41.2 | −63.5 | 59.5 | — | — | |

Current variables are evaluated on the last day.
Units of TVBIs: cfDNA(ug/mL), protein C (%), platelets (10_{^{∧}}9/L), creatine (umol/L), GCS (unitless), and lactate (mmol/L).
**The denominator for computing the percentage difference is the average of the two group means.

Using the day 1 variables from a subset of the 6 indicators, namely protein C, lactate, and creatinine, a binomial logit model may be applied for assigning patients into septic or non-septic groups. The septic and non-septic data was pooled and a binomial logit model used in which the dependent variable is the probability that a patient is septic, and the explanatory variables are the day 1 variables of protein C, lactate, and creatinine. It was found that all estimated coefficients were significantly different from zero, and that their joint predictive power was moderately high, with AUC=0.67 (CI: 0.63 to 0.71). In general, patients with lower protein C, lower lactate, and higher creatinine were more likely to be septic patients.

### Example 5—Threshold Probabilities for Achieving Various Objectives about Sensitivity, Specificity, Positive Predictive Value, and Negative Predictive Value

For septic patients, the threshold probabilities of dying within 28 days for achieving some chosen objectives about sensitivity, specificity, PPV (Positive Predictive Value), and NPV (Negative Predictive Value) are shown in Table 10. For example, the threshold probability of 0.227 can achieve the best balance between sensitivity and specificity at 0.82, whereas the threshold probability of 0.632 can achieve the best balance between PPV and NPV at 0.86.

TABLE 10

| | | | | | | |
|---|---|---|---|---|---|---|
| THRESHOLD | | | SENSITIVITY | | | |
| PROBABILITY | SENSITIVITY | SPECIFICITY | SPECIFICITY | PPV | NPV | PPV + NPV |

(A) The Outcome resulting from setting SENSITIVITY at about 0.90

| | | | | | | |
|---|---|---|---|---|---|---|
| 0.164 | 0.906 | 0.644 | 1.550 | 0.445 | 0.956 | 1.401 |

(B) The Outcome resulting from setting PPV at about 0.90

| | | | | | | |
|---|---|---|---|---|---|---|
| 0.662 | 0.482 | 0.985 | 1.468 | 0.911 | 0.858 | 1.769 |

(C) The Outcome resulting from maximizing (Sensitivity + Specificity)

| | | | | | | |
|---|---|---|---|---|---|---|
| 0.281 | 0.824 | 0.826 | 1.649 | 0.598 | 0.937 | 1.535 |

(D) The Outcome resulting from maximizing (PPV + NPV)

| | | | | | | |
|---|---|---|---|---|---|---|
| 0.877 | 0.329 | 1.000 | 1.329 | 1.000 | 0.826 | 1.826 |

(E) The Outcome resulting from making Sensitivity to be nearly equal to Specificity

| | | | | | | |
|---|---|---|---|---|---|---|
| 0.277 | 0.824 | 0.822 | 1.646 | 0.593 | 0.937 | 1.530 |

(F) The Outcome resulting from making PPV to be nearly equal to NPV

| | | | | | | |
|---|---|---|---|---|---|---|
| 0.632 | 0.518 | 0.974 | 1.492 | 0.863 | 0.865 | 1.728 |

### Example 6—Predictive Power Compared with Clinical Scoring Systems or Using Other TVBIs and Contextual Variables

The predictive power of this risk assessment tool was greater than those of APACHE II, MODS, or SOFA. For both MODS and SOFA, the day 1 and change variables were created to assess the predictive power of each of them in the CLOGLOG model that contained the same set of contextual variables as in this model. For APACHE II, only the day 1 variable was created because APACHE II was designed to measure disease severity within 24 hours of ICU admission. As shown in Table 11, the values of AUC are 0.802 (95% CI, 0.746-0.858) for MODS, 0.862 (95% CI, 0.817-0.907) for SOFA, and 0.774 (CI: 0.723-0.826) for APACHE II. All three were lower than the AUC achieved by the day 1 specification of the present model: 0.903 (95% CI, 0.864-0.941). Although the 6 components in MODS and in SOFA are similar, SOFA performed markedly better than did MODS. The better performance of SOFA over MODS probably reflects the fact that one of the TVBIs used in constructing SOFA involved choices of different treatments for hypotension, with worse scores for higher dosages.

TABLE 11

| Explanatory | Estimated | | Explanatory | Estimated | | Explanatory | Estimated | |
|---|---|---|---|---|---|---|---|---|
| Variable | Coefficient | p-value | Variable | Coefficient | p-value | Variable | Coefficient | p-value |
| Panel 1 | | | Panel 2 | | | Panel 3 | | |
| Intercept | −8.6334 | <.0001 | Intercept | −12.3043 | <.0001 | Intercept | −6.1984 | <.0001 |

1. LEVEL EFFECTS

| | | | | | | | | |
|---|---|---|---|---|---|---|---|---|
| MODS_Day_1 | 0.2767 | <.0001 | SOFA_Day _l | 0.4441 | <.0001 | APPACHE_Day_1 | 0.0283 | 0.0234 |

2. CHANGE EFFECTS

| | | | | | | | | |
|---|---|---|---|---|---|---|---|---|
| MODS_sim- | 0.3736 | <.0001 | SOFA_sim- | 0.4302 | <.0001 | — | — | — |
| ple_change | | | ple_change | | | | | |

3. CONTEXT

| | | | | | | | | |
|---|---|---|---|---|---|---|---|---|
| Chronic Lung | 0.7035 | 0.0012 | Chronic Lung | 0.6504 | 0.0033 | Chronic Lung | 0.5412 | 0.0122 |
| Disease | | | Disease | | | Disease | | |
| Previous Brain | 0.4411 | 0.3447 | Previous Brain | 0.0971 | 0.8360 | Previous | 0.5025 | 0.2759 |
| Injury | | | Injury | | | Brain Injury | | |
| Age | 0.0234 | 0.0018 | Age | 0.0174 | 0.0185 | Age | 0.0131 | 0.0836 |
| Duration | −0.0359 | 0.3258 | Duration | −0.1176 | 0.0028 | Duration | −0.0598 | 0.0962 |
| Log(Duration) | 0.4992 | 0.1170 | Log(Duration) | 1.4445 | <.0001 | Log(Duration) | 0.3741 | 0.2202 |

| | | | |
|---|---|---|---|
| AUC | 0.802 | 0.862 | 0.774 |
| | (CI: 0.746-0.858) | (CI: 0.817-0.907) | (CI: 0.723-0.826) |
| Sample Size | 7260 | 7202 | 7270 |
| No. of Patients | 389 | 383 | 391 |

To explore the possibility that adding more TVBIs into this assessment tool can enhance its predictive power, 4 additional TVBIs were added separately into the Day 1 Specification of the CLOGLOG model shown in Table 4. Three of the additional TVBIs are the remaining components of the MODS score (bilirubin, PaO₂/FiO₂ ratio, pressure adjusted heart rate (PAR)). Neutrophil count was also examined since neutrophils are a potential source of circulating cfDNA. The day 1 and change variables of each TVBI were entered into the model as a pair because these two variables had a strong negative correlation for each TVBI. Table 12 shows that all the variables representing these four TVBIs had p-values greater than 0.20 and hence did not have significant effects on the hazard of dying. Thus, addition of more TVBIs to the tool does not increase the predictive power of the tool.

TABLE 12

| | | | Wald | | |
|---|---|---|---|---|---|
| | Estimated | Standard | Chi- | p- | Sample |
| Explanatory Variable | Coefficient | Error | Square | value | Size |
| 1. LEVEL EFFECTS | | | | | |
| Bilirubin_day_1 | 0.0003 | 0.0043 | 0.0 | 0.9389 | 6172 |
| PaO2/FiO2_day_1 | −0.0006 | 0.0016 | 0.1 | 0.7060 | 6450 |
| PAR_day_l | 0.0003 | 0.0063 | 0.0 | 0.9677 | 5569 |
| Neutrophil_day_1 | 0.0108 | 0.0106 | 1.0 | 0.3097 | 6344 |
| 2. CHANGE EFFECTS | | | | | |
| Bilirubin_simple_ | −0.0002 | 0.0025 | 0.0 | 0.9271 | 6172 |
| change | | | | | |
| PaO2/FiO2_simple_ | −0.0021 | 0.0017 | 1.6 | 0.2118 | 6450 |
| change | | | | | |
| PAR_simple_change | −0.0031 | 0.0068 | 0.2 | 0.6540 | 5569 |
| Neutrophil_simple_ | −0.0041 | 0.0093 | 0.2 | 0.6606 | 6344 |
| change | | | | | |
| 3. CONTEXT | | | | | |
| Congestive Heart | 0.9371 | 0.3074 | 9.3 | 0.0023 | 6724 |
| Failure | | | | | |
| Ischemic Heart | 0.2013 | 0.3393 | 0.4 | 0.5530 | 6724 |
| Disease | | | | | |
| Liver Disease | −0.7826 | 0.4503 | 3.0 | 0.0822 | 6724 |
| Diabetes | −0.3235 | 0.2608 | 1.5 | 0.2148 | 6724 |
| Chronic Renal | −0.1627 | 0.3136 | 0.3 | 0.6039 | 6724 |
| Insufficiency | | | | | |
| Chronic Dialysis | 0.0254 | 0.4455 | 0.0 | 0.9545 | 6724 |
| Cancer | 0.2322 | 0.3133 | 0.5 | 0.4585 | 6724 |
| Gender (Female) | 0.4349 | 0.2469 | 3.1 | 0.0782 | 6724 |

Units:
Bilirubin (umol/L);
PAR, Pressure Adjusted Heart Rate, (beats/minute*mmHg/mmHg);
PaO2/FiO2 (PO2 in mmHg and FiO2 in %);
Neutrophil (10 e9/L).
The difference in sample size reflected the fact that different TVBIs had missing values for different subsets of patients.

The assessment of the usefulness of additional contextual variables was conducted by inserting each variable separately into the day 1 specification of the CLOGLOG model shown in Table 4. From Table 12, it was found that among the preconditions, congestive heart failure had a p-value less than 0.05. Despite its small p-value, this was not in the present model for the following reasons. First, because its predictive power overlapped with that of age, its inclusion inflated the p-value of age from 0.0822 to 0.2604 so that age would be removed from the model. Second, its inclusion actually resulted in a slight reduction in the model's AUC. Based on the daily probability of dying as the classifier, the inclusion of the dummy variable representing congestive heart failure caused the AUC to decrease from 0.865 to 0.862. It is likely that for a larger sample size, congestive heart failure would have a significant effect on the hazard of dying. The precondition of liver disease had a p-value somewhat lower than 0.10, suggesting that it might have some effect on the hazard of dying. However, its coefficient was negative. Diabetes and chronic renal insufficiency also had negative but non-significant coefficient. It is not clear whether these preconditions had spurious life-saving effects, resulting from the medications for their treatments. Ischemic heart disease, chronic dialysis, and cancer had positive coefficients but their p-values were too large to be considered for the inclusion in the model.

With respect to gender, being female appears to be associated with an elevated hazard of dying. However, its p-value is 0.0782. So far, physiological reasons for female septic patients to have a higher mortality risk than their male counterparts have not been found. No gender-specific differences in the administration of treatment (e.g. use of mechanical ventilation, vasopressors/inotropes, fluids) or in the length of stay in the ICU were found.

From the raw data of the 356 septic patients who contributed information to the input data matrix of the CLOGLOG model, it was observed that some sites and types of positive cultures were associated with relatively high crude death rates. For example, the 53 patients whose site of positive cultures was urinary tract had a crude death rate of 34%, and the 72 patients whose type of positive cultures was mixed had a crude death rate of 31%, compared with the overall crude death rate of 24%. To determine if information on the sites and types of positive cultures would enhance the predictive power of the CLOGLOG model, each site or type of positive cultures was represented by a dummy variable and added into the day 1 specification of the CLOGLOG model reported in Table 4. In terms of the values of the AUC that was based on the daily probability of dying as the classifier, it was found that the addition of each of the dummy variables representing the 8 sites and 6 types of positive cultures had little effect on enhancing the model's predictive power (Panel 1 of Table 13). Paradoxically, the values of the AUC decreased as a consequence of adding each of 5 sites of positive cultures (pleural cavity, blood, peritoneal, skin, and “other”) into the CLOGLOG model. This finding reveals that the intuitively appealing AUC is not a completely consistent measure of predictive power. Here, the complete consistency of a measure is defined as the property that adding an explanatory variable into the model can never result in a worse value for the measure.

TABLE 13

| Added Explanatory | AUC | | Rho-square | | Adjusted Rho-square | |
|---|---|---|---|---|---|---|
| Variable | Panel 1 | | Panel 2 | | Panel 3 | |
| | Value | Enhancement | Value | Enhancement | Value | Enhancement |

Site of Positive Cultures:

| | | | | | | |
|---|---|---|---|---|---|---|
| Lung | 0.865 | 0.000 | 0.217 | 0.000 | 0.179 | −0.002 |
| Pleural cavity | 0.863 | −0.002 | 0.221 | 0.004 | 0.184 | 0.002 |
| Blood | 0.864 | −0.001 | 0.221 | 0.003 | 0.183 | 0.001 |
| Urinary tract | 0.866 | 0.002 | 0.218 | 0.001 | 0.180 | −0.002 |
| G.I. | 0.865 | 0.000 | 0.217 | 0.000 | 0.180 | −0.002 |
| Peritoneal cavity | 0.863 | −0.001 | 0.219 | 0.002 | 0.181 | 0.000 |
| Skin | 0.863 | −0.001 | 0.218 | 0.001 | 0.180 | −0.001 |
| Other | 0.864 | −0.001 | 0.217 | 0.000 | 0.180 | −0.002 |

Type of Positive Cultures:

| | | | | | | |
|---|---|---|---|---|---|---|
| Gram-negative bacteria | 0.865 | 0.000 | 0.217 | 0.000 | 0.180 | −0.002 |
| Gram-positive bacteria | 0.865 | 0.000 | 0.217 | 0.000 | 0.180 | −0.002 |
| Fungus | 0.869 | 0.004 | 0.221 | 0.004 | 0.183 | 0.002 |
| Mixed | 0.866 | 0.001 | 0.218 | 0.001 | 0.180 | −0.002 |
| Viral | 0.865 | 0.001 | 0.218 | 0.001 | 0.180 | −0.001 |
| Protozoan | 0.865 | 0.001 | 0.218 | 0.000 | 0.180 | −0.002 |
| Without Additional | 0.865 | — | 0.217 | — | 0.182 | — |
| Explanatory Variable | | | | | | |

Note:
In this table, the classifier for computing the values of AUC is the daily probability of dying.
Rho-square = 1 - A/B, where A is the maximum log-likelihood of the model in question, and B is the maximum log-likelihood of the null model. The null model is the model with the intercept as the only coefficient to be estimated.
Adjusted Rho-square = 1 - (A-k-1)/(B-1), where k is the number of explanatory variables.

A completely consistent measure of predictive power for a CLOGLOG or logit model is the Rho-square, which is defined as 1−A/B, where A is the maximum log-likelihood of the model in question, and B is the maximum log-likelihood of the null model, which has the intercept as the only unknown coefficient. In panel 2 of Table 13, it can be seen that the addition of any of the dummy variables did not result in a decrease in the value of Rho-square. This complete consistency is the same as the complete consistency of the R-square for regression models. An important difference between them is that as demonstrated in Panel 2, a value of about 0.2 for Rho-square can represent a very high predictive power, whereas such a value for R-square usually indicates a low predictive power. To impose a penalty on adding explanatory variables that either contain mostly random noises or are highly redundant, the Rho-square is modified into the Adjusted Rho-square, which is 1−(A−k−1)/(B−1), where k is the number of explanatory variables. This is analogous to the Adjusted R-square for keeping regression models parsimonious. As seen in Panel 3 of Table E14, most of the sites and types of positive cultures contributed negatively to the Adjusted Rho-square and hence should not be added to the CLOGLOG model as these had little effect on its predictive power.

### Example 7—Mortality Risk Profiles for Individual Septic Patients

In addition to generating a predicted probability for each patient (as a survivor or non-survivor), this assessment tool can generate personalized mortality risk profiles that provide information about how different TVBIs affect a patient's risk of dying on any given day. To identify the main TVBIs that contribute to mortality risk and to determine how improvements in TVBIs can reduce the mortality risk of a patient in question, the construction and use of a mortality risk profile is described. As a basis for constructing a mortality risk profile, the best 10th percentile of survivor patients in terms of the predicted probability of dying as of the last day served as the benchmark for comparison.

Based on the estimated coefficients of the day 1 specification of the CLOGLOG model, the construction of the mortality risk profile is demonstrated for a 66-year old male patient who remained alive on day 28 (Patient A). The primary data of Patient A and the benchmark for constructing the mortality risk profile are shown in Table 14.

TABLE 14

| Variables | Patient in Question | Benchmark | Difference |
|---|---|---|---|
| A. Day 1 Variables | | | |
| cfDNA (ug/mL) | 6.9 | 4.3 | 1.8 |
| Protein C (% of normal) | 33 | 80.5 | −47.5 |
| Platelets (10\hat{ }9/L) | 119 | 221.9 | −102.9 |
| Creatinine (umol/L) | 188 | 168.3 | 19.7 |
| GCS (3-15) | 3 | 9.0 | −6.0 |
| Lactate (mmol/L) | 12.4 | 4.5 | 7.9 |
| B. Current Variables | | | |
| cfDNA (ug/mL) | 3.9 | 4.0 | −0.1 |
| Protein C (% of normal) | 47 | 131.1 | −84.1 |
| Platelets (10\hat{ }9/L) | 111 | 309.1 | −198.1 |
| Creatinine (umol/L) | 146 | 83.3 | 62.7 |
| GCS (3-15) | 11 | 14.4 | −3.4 |
| Lactate (mmol/L) | 2.3 | 1.3 | 1.0 |
| C. Context | | | |
| Chronic Lung Disease | 0 | 0.1 | −0.1 |
| Previous Brain Injury | 0 | 0.0 | 0.0 |
| Age | 66 | 54.0 | 12.0 |
| Duration | 28 | 20.3 | 7.7 |

Patient ID = 11106 (male)
For each TVEBI, the change variable is to be computed from the corresponding day 1 and current variables.

In Table 15, these data are then used to generate the values of all explanatory variables for both Patient A (in the 2nd numeric column) and the benchmark (in the 4th numeric column). For example, the value of the explanatory variable “Log(ProteinC_change)” for Patient A is generated from his day 1 and current values of protein C (33 and 47) as log(47/33)=0.35, where log is the natural log function. The remaining computations in the table are identical to those used in Table 7.

TABLE 15

| | | | Additive | | Additive | | |
|---|---|---|---|---|---|---|---|
| | | Values of | Contribution | Mean of | Contribution | Values of | Mortality |
| Explanatory | Estimated | Explanatory | to log of | the Best | to log of | Explanatory | Risk |
| Variable | Coefficient | Variable | hazard | Decile | hazard | Variable | Profile |
| | | Patient A | | The Benchmark | | Difference | |
| | Vector ″B″ | Vector ″X″ | Terms in B′X | Vector ″X″ | Terms in B′X | Vector ″X″ | Terms in B′X |
| Intercept | −1.6867 | 1 | −1.687 | 1 | −1.687 | | |
| cfDNA_day_1 | 0.1857 | 6.1 | 1.133 | 4.28 | 0.795 | 1.8 | 0.34 |
| Log(ProteinC_day_1) | −0.7289 | 3.50 | −2.549 | 4.39 | −3.199 | −0.89 | 0.65 |
| Log(Platelets_day_1) | −0.4426 | 4.78 | −2.115 | 5.40 | −2.391 | −0.62 | 0.28 |
| Log(Creatinine_day_1) | 0 | 5.24 | 0.000 | 2.23 | 0.000 | 3.01 | 0.00 |
| GCS_day_1 | −0.1281 | 3 | −0.384 | 9 | −1.157 | −6 | 0.77 |
| Lactate_day _l | 0.0661 | 12.4 | 0.820 | 4.5 | 0.296 | 7.9 | 0.52 |
| cfDNA_simple_change | 0.1871 | −2.2 | −0.412 | −0.3 | −0.049 | −1.9 | −0.36 |
| Log(ProteinC_change) | −0.6341 | 0.35 | −0.224 | 0.49 | −0.309 | 0 | 0.08 |
| Log(Platelets_change) | −0.3386 | −0.07 | 0.024 | 0.33 | −0.112 | 0 | 0.14 |
| Log(Creatinine_change) | 0.5919 | −0.25 | −0.150 | −0.70 | −0.416 | 0.45 | 0.27 |
| GCS_simple_change | −0.1518 | 8 | −1.214 | 5 | −0.808 | 3 | −0.41 |
| Log(Lactate_change) | 0.7902 | −1.68 | −1.331 | −1.20 | −0.950 | −0.48 | −0.38 |
| Chronic Lung Disease | 1.021 | 0 | 0.000 | 0 | 0.073 | 0 | −0.07 |
| Brain_Injury | 1.1211 | 0 | 0.000 | 0 | 0.000 | 0 | 0.00 |
| Age | 0.0152 | 66 | 1.003 | 54 | 0.820 | 12 | 0.18 |
| Duration | −0.0666 | 28 | −1.865 | 20 | −1.351 | 8 | −0.51 |
| Log(Duration) | 1.1033 | 3.3322 | 3.676 | 3.0099 | 3.321 | 0.3223 | 0.36 |

| | | | |
|---|---|---|---|
| Predicted Log(Hazard) = B′X | −5.275 | −7.125 | 1.849 |
| Predicted Hazard = EXP(B′X) | 0.0051 | 0.0008 | 0.0043 |
| Predicted Daily Probability of Dying | 0.0051 | 0.0008 | 0.0043 |
| Predicted Probability of Dying in 28 Days | 0.133 | 0.022 | 0.111 |
| Predicted Overall Hazard Ratio | | | 6.4 |

Patient ID = 11106 (male)

The elements in the mortality risk profile (last column of Table 15) are the predictive powers of the explanatory variables. For example, the predictive power of the day 1 variable of cfDNA is 0.34, which is this variable's contribution to [(the log of hazard of Patient A)−(the log of hazard of the benchmark)]. The greater the predictive power, the greater is the variable's ability to account for the predicted overall mortality gap between Patient A and the benchmark.

The bottom 5 elements of the last column of Table 15 are alternative measures of the overall mortality gap between Patient A and the benchmark. The overall difference in log of hazard (1.849) is the sum of the predictive powers of all explanatory variables. The HR representing the mortality gap is 6.4. The predicted probability of dying in 28 days (P28) is 13.3% for Patient A and 2.2% for the benchmark, so that the gap in P28 was 11.1%.

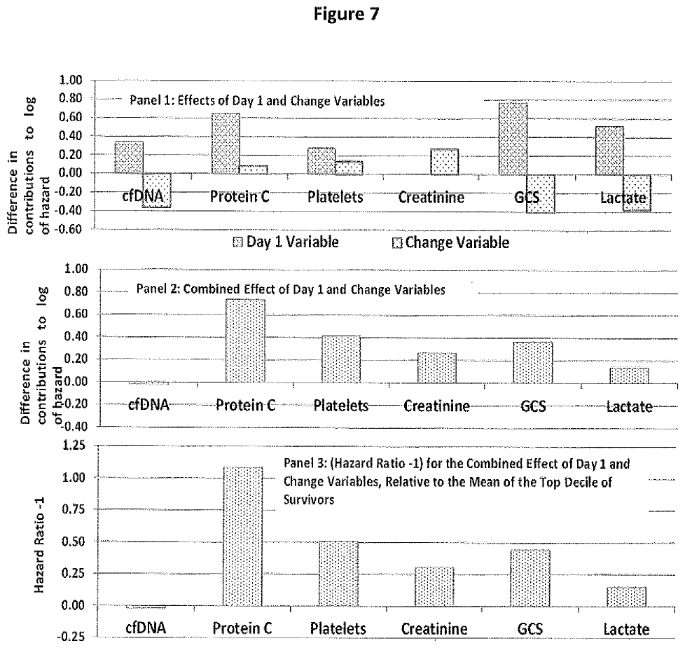

The elements in the last column of Table 15 are used to create the top graph in FIG. 7. Patient A's mortality risk was mainly due to his relatively poor values of the day 1 variables of 5 TVBIs. For cfDNA, GCS and lactate, Patient A actually experienced greater improvements than did the benchmark. In other words, if Patient A had not experienced these greater improvements, the mortality gap in P28 would have been greater than 11.1%.

By summing up the predictive powers of the day 1 and change variables of each TVBI, the combined predictive power of each TVBI was obtained as shown in the middle graph of FIG. 7, which shows three ways that the patient's mortality risk profile can be visualized. Panel 1 shows the separate effects of day 1 and change variables of each TVBI in terms of the difference in log of hazard from the benchmark. Relative to the benchmark, the patient had a higher risk of death that is mainly attributable to his unfavorable levels of GCS (contributing 0.77 to the difference), protein C (0.65), lactate (0.52), cfDNA (0.34), and platelets (0.28) on day 1. However, the improvements in GCS, lactate, and cfDNA between day 1 and day 28 helped to reduce the difference in log of hazard markedly by GCS (−0.41), lactate (−0.38) and cfDNA (−0.36), although this was offset by some worsening attributable to changes in creatinine (0.27), platelets (0.14), and protein C (0.08) relative to the benchmark. Panel 2 shows the net effect of the day 1 and change variables for each TVBI (ie. the sum of the “Day 1 variable” bar and the “Change variable” bar in Panel 1).

From this graph, it is seen that Patient A's improvements in GCS and lactate were not large enough to fully compensate for the initial disadvantage, although the improvement in his cfDNA was able to do so. For ease of communication, the exponential transformation to each bar in the middle graph was obtained to make it into a hazard ratio (HR). For ease of visualization, (HR−1) was plotted for each TVBI in the bottom graph of FIG. 7 since hazard ratios (HRs) are easier to interpret than differences in the log of hazard, the latter measures were converted into the former measures by exponential transformation. The HRs used to create this graph are multiplicative components of the overall HR of 6.4. Among these components, the HR for protein C (2.1) was the greatest followed by 1.51 for platelets, and 1.44 for GCS. The pattern suggests that abnormalities in protein C, platelets, and GCS are the major contributors to this patient's risk of dying, further suggesting that therapeutic strategies to increase protein C levels would be beneficial to Patient A.

Using the dynamic Excel version of Table 15, it was found that by increasing Patient A's protein C (47) to the level of the benchmark (131), his P28 could be decreased from 13.3% to 7.2%. FIG. 7 suggests that further reduction of his P28 should focus on improving his platelets and GCS. It was found that by increasing his platelets (111) and GCS (11) to the levels of the benchmark (309 and 14, respectively), his P28 could be further reduced to 3.3%.

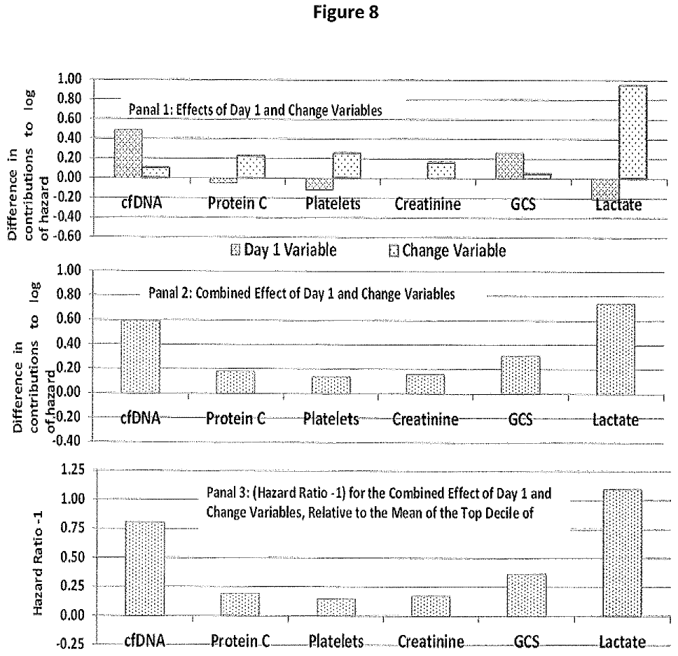

To demonstrate how the knowledge of the dynamic nature of the benchmark is useful in assisting the identification of the TVBIs that are a high priority for improvement, the mortality risk profile of another male patient (Patient B) who was discharged on day 12 was constructed. The primary data of Patient B and the benchmark are shown in Table 16, the computations are presented in Table 17, and his mortality risk profile is shown in FIG. 8.

TABLE 16

| Variables | Patient in Question | Benchmark | Difference |
|---|---|---|---|
| A. Day 1 Variables | | | |
| cfDNA (ug/mL) | 6.9 | 4.3 | 2.6 |
| Protein C (% of normal) | 86 | 80.5 | 5.5 |
| Platelets (10\hat{ }9/L) | 290 | 221.9 | 68.1 |
| Creatinine (umol/L) | 68 | 168.3 | −100.3 |
| GCS (3-15) | 7 | 9.0 | −2.0 |
| Lactate (mmol/L) | 1.3 | 4.5 | −3.2 |
| B. Current Variables | | | |
| cfDNA (ug/mL) | 7.2 | 4,0 | 3.2 |
| Protein C (% of normal) | 98 | 131.1 | −33.1 |
| Platelets (10\hat{ }/L) | 190 | 309.1 | −119.1 |
| Creatinine (urriol/L) | 44 | 83.3 | −39.3 |
| GCS (3-15) | 12 | 14.4 | −2.4 |
| Lactate (mmol/L) | 1.3 | 1.3 | 0.0 |
| C. Context | | | |
| Chronic Lung Disease | 1 | 0.1 | 0.9 |
| Previous Brain Injury | 0 | 0.0 | 0.0 |
| Age | 79.2 | 54.0 | 25.2 |
| Duration | 12 | 20.3 | −8.3 |

Patient ID = 61013 (male)
For each TVBI, the change variable is to be computed from the corresponding day 1 and current variables,

TABLE 17

| | | | Additive | | Additive | | |
|---|---|---|---|---|---|---|---|
| | | Values of | Contribution | Mean of | Contribution | Values of | Mortality |
| Explanatory | Estimated | Explanatory | to log of | the Best | to log of | Explanatory | Risk |
| Variable | Coefficient | Variable | hazard | Decile | hazard | Variable | Profile |
| | | Patient B | | The Benchmark | | Difference | |
| | Vector ″B″ | Vector ″X″ | Terms in B′X | Vector ″X″ | Terms in B′X | Vector ″X″ | Terms in B′X |
| Intercept | −1.6867 | 1 | −1.687 | 1 | −1.687 | | |
| cfDNA_day_1 | 0.1857 | 6.9 | 1.281 | 4.28 | 0.795 | 2.6 | 0.49 |
| Log(ProteinC_day_1) | −0.7289 | 4.45 | −3.247 | 4.39 | −3.199 | 0.07 | −0.05 |
| Log(Platelets_day_1) | −0.4426 | 5.67 | −2.509 | 5.40 | −2.391 | 0.27 | −0.12 |
| Log(Creatinine_day_1) | 0 | 4.22 | 0.000 | 2.23 | 0.000 | 1.99 | 0.00 |
| GCS_day_1 | −0.1281 | 7 | −0.897 | 9 | −1.157 | −2 | 0.26 |
| Lactate_day _l | 0.0661 | 1.3 | 0.086 | 4.5 | 0.296 | −3.2 | −0.21 |
| cfDNA_simple_change | 0.1871 | 0.3 | 0.056 | −0.3 | −0.049 | 0.6 | 0.10 |
| Log(ProteinC_change) | −0.6341 | 0.13 | −0.083 | 0.49 | −0.309 | 0 | 0.23 |
| Log(Platelets_change) | −0.3386 | −0.42 | 0.143 | 0.33 | −0.112 | −1 | 0.26 |
| Log(Creatinine_change) | 0.5919 | −0.44 | −0.258 | −0.70 | −0.416 | 0.27 | 0.16 |
| GCS_simple_change | −0.1518 | 5 | −0.759 | 5 | −0.808 | 0 | 0.05 |
| Log(Lactate_change) | 0.7902 | 0.00 | 0.000 | −1.20 | −0.950 | 1.20 | 0.95 |
| Chronic Lung Disease | 1.021 | 1 | 1.021 | 0 | 0.073 | 1 | 0.95 |
| Brain_Injury | 1.1211 | 0 | 0.000 | 0 | 0.000 | 0 | 0.00 |
| Age | 0.0152 | 79.2 | 1.204 | 54 | 0.820 | 25 | 0.38 |
| Duration | −0.0666 | 12 | −0.799 | 20 | −1.351 | −8 | 0.55 |
| Log(Duration) | 1.1033 | 2.4849 | 2.742 | 3.0099 | 3.321 | −0.5250 | −0.58 |

| | | | |
|---|---|---|---|
| Predicted Log(Hazard) = B′X | −3.705 | −7.125 | 3.419 |
| Predicted Hazard = EXP(B′X) | 0.0246 | 0.0008 | 0.0238 |
| Predicted Daily Probability of Dying | 0.0243 | 0.0008 | 0.0235 |
| Predicted Probability of Dying in 28 Days | 0.498 | 0.022 | 0.475 |
| Predicted Overall Hazard Ratio | | | 30.55 |

Patient ID = 61013 (male)

Partly as a consequence of having an advanced age of 79 and the precondition of chronic lung disease, his P28 was quite high (50%). The finding that among the 6 TVBIs, lactate had the highest HR of 2.1 suggests that lowering his lactate level would be most effective for reducing his mortality risk. However, a closer examination of Tables 16 and 17 revealed that Patient B started with a very low level of lactate (1.3 mmol/L) and maintained the same level on the day of discharge, whereas the benchmark started with a rather high lactate level (4.5) and experienced a large decrease (−3.2) to a low level (1.3) on the last day. Thus, the impressive improvement of the benchmark's lactate could not be replicated by Patient A, because his lactate level was already very low.

In FIG. 8, cfDNA had the next highest HR (1.8). From Table 16, it is seen that the level of cfDNA on the last day was much higher for Patient 13 (7.2 ug/mL) than for the benchmark (4.0 μg/mL). Using the Excel version of Table 17 it was found that by reducing Patient B's cfDNA to 4.0 μg/mL and 2.0 μg/mL, his P28 was reduced from 50% to 32% and 23%, respectively. With an HR of 1.36, GCS was the next TVBI to be considered for improvement. Together with a reduction of cfDNA to 2.0, the increase of GCS from the current level of 12 to 15 would further reduce Patient B's P28 to 15%.

### Example 8—Validation of the Risk Assessment Tool

To validate the CLOGLOG model, the estimated coefficients of the day 1 specification of the model were used to predict the mortality outcomes of ICU patients from nine Canadian hospitals who were originally non-septic but later became septic in the ICU. Of the 33 such patients recruited from the same ICUs and during the sample time frame as the septic patients, 28 had non-missing values for all the explanatory variables and hence were used to form the validation group.

Before conducting the validation, the issue about what should be the definition of day 1: (1) the day of admission or (2) the day of becoming septic was considered. It was decided to conduct two validations: (V1) with the admission date as day 1 and (V2) with the day of becoming septic as day 1. For V2, the censored date was extended for any patient who was still alive on day 28 until he/she died, discharged, or remained alive on day 28 since becoming septic. Since there were 5 non-survivors in V1 and 6 non-survivors in V2, the proportion of non-survivors was 18% in V1 and 21% in V2.

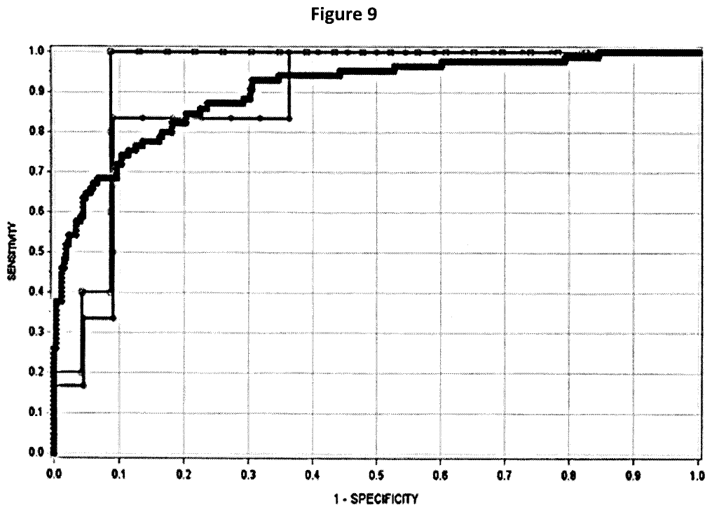

For each patient in the validation group, the values of all explanatory variables as of her/his last day were first found and then her/his predicted hazard of dying was computed by inputting these values into the estimated day 1 specification of the CLOGLOG model. The predicted hazards of all 28 patients were then translated into the predicted probabilities of dying in 28 days for conducting an ROC analysis. The resulting AUC turned out to be quite high: 0.939 (95% CI, 0.845-1.000) for V1 and 0.886 (95% CI, 0.746-1.000) for V2, compared with 0.903 (95% CI, 0.864-0.941) for the derivation group of 356 patients. Since the validation group had a much smaller sample size than did the derivation group, the 95% confidence intervals of the AUCs for V1 and V2 were much wider than that for the derivation group. Despite the smaller sample size, the validation results were statistically supportive, because the lower limits of 0.845 and 0.746 were much higher than 0.5. The ROC curves for the V1 and V2 validations are compared with that of the derivation group in FIG. 9.

In Table 18, the average values of the six TVBIs on day 1 and on the last day, as well as the change between the two dates, are shown separately for the non-survivors and survivors in V1 and V2. The contrasts between non-survivors and survivors were more consistent to the expected effects on the last day than on day 1 in both V1 and V2. The expectation was that for lactate, cfDNA, and creatinine, the means should be higher for the non-survivors than for the survivors, whereas for GCS, protein C, and platelets, the opposite would occur. With respect to the change from day 1 to the last day, the difference between non-survivors and survivors was consistent to the expectation for platelets, GCS, and lactate but inconsistent for cfDNA, protein C, and creatinine. In both V1 and V2, the consistent changes were greater than the inconsistent changes.

TABLE 18

| | Non-survivors | Survivors | Difference | Non-survivors | Survivors | Difference |
|---|---|---|---|---|---|---|
| TVBI | Panel 1: V1 (Day 1 = Admission) | | | Panel 2: V2 (Day 1 = Becoming Septic) | | |
| | On Day 1 | | | On Day 1 | | |
| cfDNA (ug/mL) | 4.24 | 3.85 | 0.39 | 3.92 | 3.92 | 0.00 |
| Protein C (% of normal) | 72.8 | 83.5 | −10.7 | 81.2 | 81.7 | −0.5 |
| Platelets (10_{^{∧}}9/L) | 231.2 | 191.6 | 39.6 | 229.8 | 190.1 | 39.7 |
| Creatinine (umol/L) | 326.2 | 106.5 | 219.7 | 286.7 | 107.3 | 179.4 |
| GCS (3-15) | 8.00 | 7.78 | 0.22 | 7.50 | 7.91 | −0.41 |
| Lactate (mmol/L) | 2.48 | 3.77 | −1.29 | 2.55 | 3.80 | −1.25 |
| | On the Last Day | | | On the Last Day | | |
| cfDNA (ug/mL) | 4.50 | 4.12 | 0.38 | 4.13 | 4.21 | −0.08 |
| Protein C (% of normal) | 93.9 | 100.4 | −6.5 | 102.1 | 98.5 | 3.6 |
| Platelets (10_{^{∧}}9/L) | 205.4 | 324.7 | −119.3 | 188.2 | 334.8 | −146.6 |
| Creatinine (umol/L) | 286.6 | 120.8 | 165.8 | 247.8 | 123.9 | 124.0 |
| GCS (3-15) | 8.60 | 11.87 | −3.27 | 8.17 | 12.14 | −3.97 |
| Lactate (mmol/L) | 5.72 | 1.49 | 4.23 | 5.05 | 1.48 | 3.57 |
| | Change from day 1 to the last day (%) | | | Change from day 1 to the last day (%) | | |
| cfDNA | 6.1 | 7.0 | −0.9 | 5.4 | 7.4 | −2.0 |
| Protein C | 28.9 | 20.3 | 8.7 | 25.7 | 20.6 | 5.2 |
| Platelets | −11.2 | 69.5 | −80.6 | −18.1 | 76.1 | −94.2 |
| Creatinine | −12.1 | 13.5 | −25.6 | −13.5 | 15.5 | −29.0 |
| GCS | 7.5 | 52.6 | −45.1 | 8.9 | 53.5 | −44.5 |
| Lactate | 130.6 | −60.5 | 191.1 | 98.0 | −61.1 | 159.1 |
| No. of Patients | 5 | 23 | | 6 | 22 | |

In terms of the contributions to the difference in log of mortality hazard between non-survivors and survivors, the contributions of lactate (1.30) and GCS (0.50) were much greater than those of platelets (0.14), cfDNA (0.07), protein C (0.06), and creatinine (−0.15) in V1. Similarly, the contributions of lactate (1.19) and GCS (0.56) were also much greater than those of platelets (0.17), cfDNA (0.00), protein C (−0.01), and creatinine (−0.16) in V2. Thus, the validation group confirmed that among the six TVBIs, lactate and GCS, mostly through their change effects, had the strongest predictive powers, irrespective of whether day 1 was defined as the date of admission or the day of becoming septic.

### Example 9—Qualitatively Similar Insights from the Analogous Longitudinal Logit Model

To demonstrate that the longitudinal logit (L-Logit) model specified in Eq. 5 could be used to reveal similar insights as those obtained via the CLOGLOG model, it was used to create the mortality risk profile of Patient A. With the input data matrix of the day 1 specification of the CLOGLOG model, the coefficients of the L-Logit model were estimated by simply replacing “LINK=CLOGLOG” by “LINK=LOGIT” in the Logistic procedure of SAS. With AUC=0.904 (95% CI, 0.865-0.942), its predictive power was about the same as that of the CLOGLOG model.

Analogous to the predicted log of hazard in the CLOGLOG model, the predicted logit in the L-Logit model can be decomposed into additive terms in the following way:

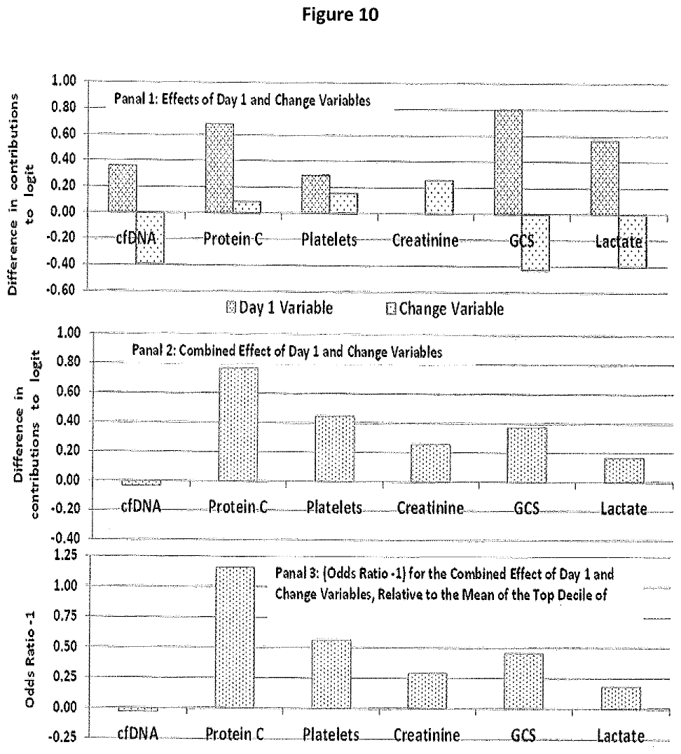

G̅ᵢₜ=log(P̅ᵢₜ/(1−P̅ᵢₜ))=β̅₀+β̅₁Xᵢ₁ₜ+β₂Xᵢ₂ₜ+ . . . +β̅ₖXᵢₖₜ+ . . . (6)

where G̅ᵢₜ is the ith patient's predicted logit of dying on day t, P̅ᵢₜ is the ith patient's predicted probability of dying on day t, and β̅ₖXᵢₖₜ is the additive contribution of the kth explanatory variable to the predicted logit. With the estimated coefficients (in the first numeric column of Table 19), the additive contributions to the predicted logits of (1) Patient A and (2) the benchmark (in the 3^{rd} and 5^{th} columns of Table 19) was computed. The values of the difference in the contributions to the predicted logits between (1) and (2) were then computed (in the last column of Table 19). These values are interpreted as the predictive powers of the explanatory variables in accounting for the patient's mortality risk relative to the benchmark that represented the mean of the best decile of the survivors. The predictive powers attributable to the day 1 and change variables of each of the 6 TVBIs were plotted in Panel 1 of FIG. 10. The combined predictive power of the day 1 and change variables of each TVBI was then plotted in Panel 2 of FIG. 10. The predictive powers (the differences in logit) were then translated to the corresponding odds ratios by exponentiation and then the values of (odds ratio−1) were plotted in Panel 3 of FIG. 10, 11 should not be surprising that the patterns of the bars in the 3 panels of FIG. 10 are very similar to those in FIG. 7, because logit and odds ratio in the L-Logit model are analogous to log of hazard and hazard ratio in the CLOGLOG model.

TABLE 19

| | | Values of | Additive | Mean of | Additive | Values of | Mortality |
|---|---|---|---|---|---|---|---|
| Explanatory | Estimated | Explanatory | Contribution | the Best | Contribution | Explanatory | Risk |
| Variable | Coefficient | Variable | to logit | Decile | to logit | Variable | Profile |
| | | Patient A | | The Benchmark | | Difference | |
| | Vector ″B″ | Vector ″X″ | Terms in B′X | Vector ″X″ | Terms in B′X | Vector ″X″ | Terms in B′X |
| Intercept | −1.5941 | 1 | −1.594 | 1 | −1.594 | | |
| cfDNA_day_1 | 0.1975 | 6.1 | 1.205 | 4.28 | 0.845 | 1.8 | 0.36 |
| Log(ProteinC_day_1) | −0.7635 | 3.50 | −2.670 | 4.39 | −3.350 | −0.89 | 0.68 |
| Log(Platelets_day_1) | −0.4686 | 4.78 | −2.239 | 5.40 | −2.531 | −0.62 | 0.29 |
| Log(Creatinine_day_1) | 0 | 5.24 | 0.000 | 2.23 | 0.000 | 3.01 | 0.00 |
| GCS_day_1 | −0.1328 | 3 | −0.398 | 9 | −1.200 | −6 | 0.80 |
| Lactate_day _l | 0.0716 | 12.4 | 0.888 | 4.5 | 0.321 | 7.9 | 0.57 |
| cfDNA_simple_change | 0.2024 | −2.2 | −0.445 | −0.3 | −0.053 | −1.9 | −0.39 |
| Log(ProteinC_change) | −0.6565 | 0.35 | −0.232 | 0.49 | −0.320 | 0 | 0.09 |
| Log(Platelets_change) | −0.3866 | −0.07 | 0.027 | 0.33 | −0.128 | 0 | 0.16 |
| Log(Creatinine_change) | 0.5676 | −0.25 | −0.144 | −0.70 | −0.399 | 0.45 | 0.26 |
| GCS_simple_change | −0.1587 | 8 | −1.270 | 5 | −0.845 | 3 | −0.43 |
| Log(Lactate_change) | 0.831 | −1.68 | −1.400 | −1.20 | −0.999 | −0.48 | −0.40 |
| Chronic Lung Disease | 1.1072 | 0 | 0.000 | 0 | 0.079 | 0 | −0.08 |
| Brain_Injury | 1.1502 | 0 | 0.000 | 0 | 0.000 | 0 | 0.00 |
| Age | 0.0165 | 66 | 1.089 | 54 | 0.890 | 12 | 0.20 |
| Duration | −0.0691 | 28 | −1.935 | 20 | −1.402 | 8 | −0.53 |
| Log(Duration) | 1.1383 | 3.3322 | 3.793 | 3.0099 | 3.426 | 0.3223 | 0.37 |

| | | | |
|---|---|---|---|
| Predicted Logit = B′X | −5.325 | −7.260 | 1.935 |
| Predicted Odds = EXP(B′X) | 0.0049 | 0.0007 | 0.0042 |
| Predicted Daily Probability of Dying | 0.0048 | 0.0007 | 0.0041 |
| Predicted Probability of Dying in 28 Days | 0.127 | 0.019 | 0.108 |
| Predicted Overall Odds Ratio | | | 6.9 |

### DISCUSSION

A tool for assessing the mortality risk in septic patients has been developed. At the core of the tool was a CLOGLOG model that created a composite indicator from six TVBIs (cfDNA, protein C, lactate, GCS, platelets, and creatinine) and some contextual variables (age, presence of chronic lung disease or previous brain injury, length of stay). With the same set of contextual variables, the set of six TVBIs was stronger in predictive power (AUC-0.90) than not only APACHE II (AUC-0.77) but also MODS (AUC=0.80) and SOFA (AUC=0.86), both of which were also constructed from six TVBIs. The demonstration that both day 1 and change variables are important helps explain why APACHE II, which was based on the observed values of TVBIs in the first 24 hours, had the weakest predictive power.

There are several strengths to the present study. First, the way of formulating the CLOGLOG model made the model a versatile version of the Cox model for gaining longitudinal insights. The versatility comes from the removal of the restrictive assumption of proportional hazards and the replacement of the maximum partial-likelihood method by the maximum likelihood method for estimation. With the alternative estimation method, a flexible time function was used that helped overcome a distortion resulting from selection bias. Second, the present tool can generate mortality risk profiles that show the relative contributions of the six TVBIs to each patient's overall mortality risk. For example, a septic patient whose risk was mainly due to deficiencies in protein C may benefit from therapies that enhance the conversion of protein C to the anticoagulant activated protein C (APC). Potential therapies include ART-123, a recombinant thrombomodulin that enhances APC generation. In a phase Ilb clinical trial, ART-123 was shown to be safe and potentially efficacious in septic patients. Patients whose risks were mainly due to elevations in cfDNA may benefit from strategies that lower cfDNA. Administration of recombinant DNasel to septic animals has been shown to reduce circulating levels of DNA, suppress organ damage, and improve survival. Third, this is the first study that describes the use of the CLOGLOG model for predicting the probability of dying on any day or in any time interval in septic patients. Extensive exploration has also been conducted to see whether adding more TVBIs (such as bilirubin, PaO₂/FiO₂ ratio, pressure adjusted heart rate) or more contextual variables (such as types and sites of infection) could improve the model. The exploration confirmed that the present TVBIs provide a robust and concise model, and the statistical indicators for confidence turned out to be quite strong.

### CONCLUSION

A tool to predict the mortality risk over time in septic patients and for generating personalized risk profiles has been developed and validated. This tool is based on a CLOGLOG model that takes advantage of the changing values of cfDNA, protein C, platelet count, creatinine, GCS, and lactate to achieve a high predictive power. The tool can help stratify patients who have similar clinical presentations but may respond differently to treatments due to patient-specific pathophysiology. The tool has utility for prognostic and predictive enrichment which may be leveraged to improve the success of clinical trials.

While the present application has been described with reference to examples, it is to be understood that the scope of the claims should not be limited by the embodiments set forth in the examples, but should be given the broadest interpretation consistent with the description as, a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A method of determining the risk of mortality in a septic patient comprising:
i) determining in a biological sample obtained from a patient the level of each of cfDNA, protein C, lactate, platelet count, creatinine, and GCS time-varying indicators, and
ii) comparing the level of each indicator from step (i) to a control or normal level, or to a previously determined level, to provide an assessment of mortality risk, wherein an elevated level of any one of cfDNA, lactate and creatinine or a lowered level of any one of protein C, platelets and GCS, as compared to the control or previously determined level, is indicative of an increased risk of death in the patient.

2. The method of claim 1, wherein there is an increased risk of death in the patient when there is an increase of at least about 1.5-fold in the level of any of cfDNA, lactate and creatinine, or a decrease in the level of protein C to ≤65% of normal levels, of platelets to ≤200×10⁹/L or a 10% decrease/day) or a decrease in GCS to ≤12.

3. The method of claim 1, wherein two or more indicator levels are indicative of increased risk of death.

4. The method of claim 1, wherein indicator levels are determined at a plurality of time-points to obtain time-varying indicator values.

5. The method of claim 1, wherein the levels of cfDNA, protein C, platelets and GCS are compared to a control or normal level, and the levels of lactate and creatinine are compared to a previously determined level of each.

6. The method of any one of claims 1-5, wherein an increase of at least about 1.5-fold in the level of any of cfDNA, lactate and creatinine, or a decrease in the level of protein C and platelets by about 1.5-fold, from normal, or a decrease in GCS, is indicative of increased risk of death in a mammal.

7. The method of claim 1, wherein the levels of the indicators are compared to control levels in exponential form, power form, or exponential and power forms to maximize the predictive power of the model.

8. The method of claim 1, wherein a subset of the indicators can be used in a binomial logit model to distinguish a septic from a non-septic patient.

9. The method of claim 7, wherein the subset of indicators comprises protein C, lactate, and creatinine.

10. The method of claim 1, wherein lactate and creatinine are measured via enzymatic digestion.

11. The method of claim 10, wherein the level of lactate is determined by digestion with lactate oxidase and the level of creatinine is determined by digestion with picric acid.

12. The method of claim 1, wherein the level of cfDNA is determined by measuring UV absorbance at 260 nm.

13. The method of claim 1, wherein the level of protein C antigen is determined using an enzyme immunoassay.

14. The method of claim 1, additionally including a step of treating the septic patient with one or a combination of treatments to reduce level of cfDNA, lactate and/or creatinine, and/or to increase level of protein C, platelet levels, and/or GCS.

15. The method of claim 14, wherein the treatment is selected from ART-123 to boost protein C levels, an anticoagulant or antiplatelet drug to inhibit blood clotting due to elevated levels of cfDNA, or an immune boosting treatment.

16. A method for determining the probability of a septic patient dying on a specific day or within a certain time frame comprising:
i) determining in a biological sample obtained from a patient the level of each of cfDNA, protein C, lactate, platelet count, creatinine, and GCS time-varying indicators, and
ii) determining the probability of dying based on a complementary log-log analysis of the levels of one or more of the time-varying indicators.

17. A method for monitoring response to treatment in a septic patient comprising:
i) determining in a biological sample obtained from a patient the baseline level of each of cfDNA, protein C, lactate, platelet count, creatinine, and GCS at the onset of treatment, and one or more treatment levels at one or more time points following onset of treatment,
ii) comparing the treatment level of each indicator to the baseline level, and providing an assessment of response to treatment, wherein a reduced level of any of cfDNA, lactate and creatinine or an increased level of any of protein C, platelets and GCS indicates that the patient is responding to treatment.

18. The method of claim 17, wherein the treatment is to reduce the level of any of cfDNA, lactate and creatinine, or to increase the level of any of protein C, platelets and GCS.

19. A method of generating a personalized mortality risk profile for a septic comprising:
i) determining in a biological sample obtained from a patient the levels of one or more of cfDNA, protein C, lactate, platelet count, creatinine, and GCS indicators over time, and
ii) determining the change in the level of the one or more indicators over time as compared with control or benchmark levels; and
iii) providing a profile of indicator levels based on a longitudinal logit (L-Logit) model or complementary log-log analysis of the change in indicator levels.
